(19) United States
(12) Patent Application Publication
(10) Pub. No.: US 2024/0294617 A1
(43) Pub. Date: Sep. 5, 2024

(54) SERUM ALBUMIN BINDERS

(71) Applicant: Ablynx N.V, Zwijnaarde (BE)

(72) Inventors: Stephanie Staelens, Gent (BE); Soren Steffensen, Zwijnaarde (BE); Erika Morizzo, Cambridge (GB); An Cerdobbel, Nevele (BE)

(73) Assignee: Ablynx N.V., Zwijnaarde (BE)

(21) Appl. No.: 18/404,145

(22) Filed: Jan. 4, 2024

Related U.S. Application Data
(62) Division of application No. 16/477,927, filed on Jul. 15, 2019, now Pat. No. 11,897,944.
(60) Provisional application No. 62/446,992, filed on Jan. 17, 2017.

(51) Int. Cl. C07K 16/18; A61K 39/00

(52) U.S. Cl. CPC C07K 16/18; A61K 2039/505; C07K 2317/22; C07K 2317/24; C07K 2317/50; C07K 2317/56; C07K 2317/569; C07K 2317/62; C07K 2317/622; C07K 2317/624; C07K 2317/626; C07K 2317/92

(57) ABSTRACT

The present invention relates to amino acid sequences that can bind to serum albumin. In particular, the present invention relates to immunoglobulin single variable domains, and in particular heavy-chain immunoglobulin single variable domains, that can bind to serum albumin. The invention also relates to proteins, polypeptides and other constructs, compounds, molecules or chemical entities that comprise at least one of the immunoglobulin single variable domains binding to serum albumin that are described herein.

### RELATED APPLICATIONS

This application is a divisional of U.S. application Ser. No. 16/477,927, filed Jul. 15, 2019, which is a national stage filing under 35 U.S.C. § 371 of International Patent Application PCT/EP2018/051082, filed Jan. 17, 2018, which claims priority under 35 U.S.C § 119(e) to U.S. Provisional Application Ser. No. 62/446,992, filed Jan. 17, 2017, the entire contents of each of which are incorporated herein in their entirety.

### REFERENCE TO AN ELECTRONIC SEQUENCE LISTING

The content of the electronic sequence listing (A084870203US02-SEQ-CRP.xml; Size: 107,383 bytes; and Date of Creation: Jan. 4, 2024) is herein incorporated by reference in its entirety.

The present invention relates to amino acid sequences that can bind to serum albumin.

In particular, the present invention relates to immunoglobulin single variable domains, and in particular heavy-chain immunoglobulin single variable domains, that can bind to serum albumin.

As described herein, the immunoglobulin single variable domains provided by the invention are preferably such that they can (at least) bind (and in particular, specifically bind) to human serum albumin. More preferably, as further described herein, these immunoglobulin single variable domains are preferably further such that they are cross-reactive (as described herein) between human serum albumin and serum albumin from at least one other species of mammal.

The invention also relates to proteins, polypeptides and other constructs, compounds, molecules or chemical entities that comprise at least one of the immunoglobulin single variable domains binding to serum albumin that are described herein.

Immunoglobulin single variable domains will also generally be referred to herein by means of the abbreviations “ISV's” or “ISVD's” (which will be used interchangeably herein).

The immunoglobulin single variable domains binding to serum albumin that are described herein will also be referred to herein as “amino acid sequences of the invention”, or “serum albumin binders of the invention”. As further described herein, the albumin binders of the invention may in particular be Nanobodies (as further described herein).

The proteins, polypeptides and other constructs, compounds, molecules or chemical entities that comprise at least one of the serum albumin binder of the invention will also referred to herein as “compounds of the invention” or as “polypeptides of the invention”. Preferably, the compounds of the invention are proteins or polypeptides, and may in particular be fusion proteins.

Other aspects, embodiments, features, uses and advantages of the invention will be clear to the skilled person based on the disclosure herein.

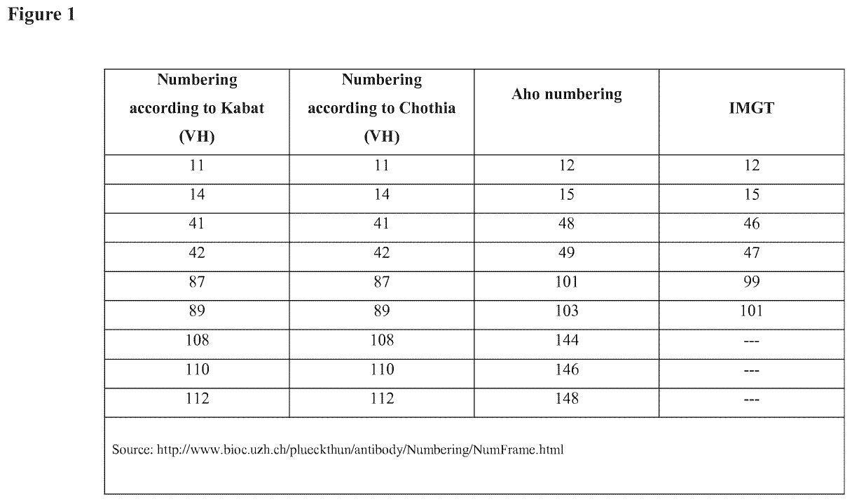

In the present application, the amino acid residues/positions in an immunoglobulin heavy-chain variable domain will be indicated with the numbering according to Kabat. For the sake of convenience, FIG. 1 gives a table listing some of the amino acid positions that will be specifically referred to herein and their numbering according to some alternative numbering systems (such as Aho and IMGT. Note: unless explicitly indicated otherwise, for the present description and claims, Kabat numbering is decisive; other numbering systems are given for reference only).

With regard to the CDR's, as is well-known in the art, there are multiple conventions to define and describe the CDR's of a VH or VHH fragment, such as the Kabat definition (which is based on sequence variability and is the most commonly used) and the Chothia definition (which is based on the location of the structural loop regions). Reference is for example made to the website bioinf.org.uk/abs/. For the purposes of the present specification and claims, even though the CDRs according to Kabat may also be mentioned, the CDRs are most preferably defined on the basis of the Abm definition (which is based on Oxford Molecular's AbM antibody modelling software), as this is considered to be an optimal compromise between the Kabat and Chothia definitions. Reference is again made to the website bioinf.org.uk/abs/).

Accordingly, in the present specification and claims, all CDRs are defined according to the Abm convention, unless explicitly stated otherwise herein.

ISVD's (and in particular Nanobodies) that can bind to serum albumin and their uses are well-known in the art, for example from WO 2004/041865, WO 2006/122787, WO 2012/175400, WO 2015/173325 and PCT/EP2016/077973, which describe serum albumin-binding ISVD's and their use for extending the serum half-life (as defined in these applications) of therapeutic compounds, moieties and entities. For example, WO 2006/122787 discloses as SEQ ID NO: 62 a humanized serum albumin-binding Nanobody called Alb-8 (see SEQ ID NO:1 herein). WO 2012/175400 discloses as SEQ ID NO: 6 a humanized serum albumin-binding Nanobody called Alb-23D (see SEQ ID NO:2 herein). The amino acid sequences of Alb-8 and Alb-23D and their CDR's (which are the same for Alb-8 and Alb-23D) are given in Table A below as SEQ ID NO: 1, 2 and 3 to 8, respectively.

Some other references that disclose ISVD's against serum albumin include WO 2003/035694, WO 2004/003019, EP 2 139 918, WO 2011/006915 and WO 2014/111550.

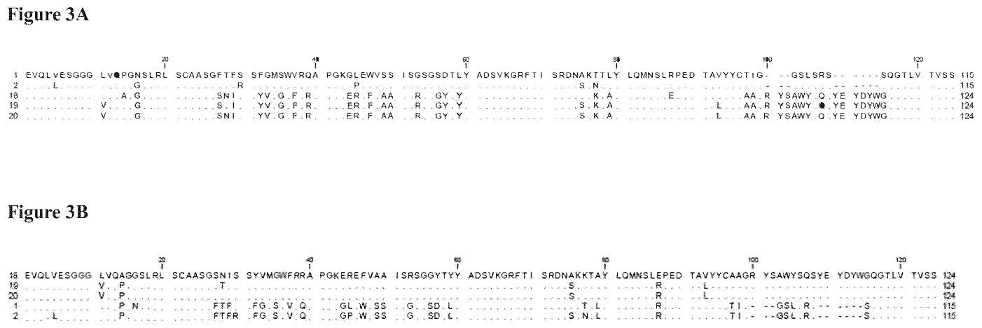

FIGS. 3A and 3B show alignments of Alb-8 (reference; SEQ ID NO: 1), Alb-23D (reference; SEQ ID NO: 2), SEQ ID NO: 18 (invention),SEQ ID NO: 19 (invention), and SEQ ID NO: 20 (invention). These sequence identifiers are also provided on the left side of the presented alignments.

The present invention aims to provide improved serum albumin binders, and in particular serum albumin binders that have improved properties compared to the serum albumin binders known in the art. In particular, the invention aims to provide serum albumin binders that can bind to dog serum albumin and/or that have improved cross-reactivity between human serum albumin and dog serum albumin (i.e. compared to prior art serum albumin binders like Alb-8 and/or Alb-23D).

TABLE A
Alb-8, Alb-23D and their CDRs

| SEQ | | |
|---|---|---|
| ID | | |
| NO | Description | Sequence |
| 1 | Alb-8 | EVQLVESGGGLVQPGNSLRLSCAASGFTF |
| | (WO 2006/122787; | SSFGMSWVRQAPGKGLEWVSSISGSGSDT |
| | SEQ ID NO: 62) | LYADSVKGRFTISRDNAKTTLYLQMNSLR |
| | | PEDTAVYYCTIGGSLSRSSQGTLVTVSS |
| 2 | Alb-23D | EVQLLESGGGLVQPGGSLRLSCAASGFTF |
| | (WO 2012/175400; | RSFGMSWVRQAPGKGPEWVSSISGSGSDT |
| | SEQ ID NO: 6) | LYADSVKGRFTISRDNSKNTLYLQMNSLR |
| | | PEDTAVYYCTIGGSLSRSSQGTLVTVSSA |
| 3 | CDR1 (Kabat) | SFGMS |
| 4 | CDR2 (Kabat) | SISGSGSDTLYADSVKG |
| 5 | CDR3 (Kabat/Abm) | GGSLSR |
| 6 | CDR1 (Abm) | GFTFRSFGMS |
| 7 | CDR2 (Abm) | SISGSGSDTL |
| 8 | CDR3 (Kabat/Abm) | GGSLSR |

Note:
SEQ ID NOs: 1 and 2 share the same CDRs according to Kabat. However, if the CDRs are defined under the Abm convention, SEQ ID NO: 1 has a different CDR1 from SEQ ID NOs: 2 compared to SEQ ID NOs: 2, SEQ ID NO: 1 has an S at position 30 instead of an R.
SEQ ID NO: 5 and SEQ ID NO: 8 are identical.
all CDRs are defined according to the Abm convention, unless indicated otherwise.

TABLE B
SEQ ID NOs: 18 and 19 and their CDR's

| SEQ | | |
|---|---|---|
| ID | | |
| NO | Description | Sequence |
| 18 | T0235005G01 | EVQLVESGGGLVQAGGSLRLSCAASGSNI |
| | | SSYVMGWFRRAPGKEREFVAAISRSGGYT |
| | | YYADSVKGRFTISRDNAKKTAYLQMNSLE |
| | | PEDTAVYYCAAGRYSAWYSQSYEYDYWGQ |
| | | GTLVTVSS |
| 19 | T023500043 | EVQLVESGGGVVQPGGSLRLSCAASGSTI |
| | | SSYVMGWFRRAPGKEREFVAAISRSGGYT |
| | | YYADSVKGRFTISRDNSKKTAYLQMNSLR |
| | | PEDTALYYCAAGRYSAWYSQSYEYDYWGQ |
| | | GTLVTVSS |
| 9 | CDR1 (Kabat) | SYVMG |
| 10 | CDR2 (Kabat) | AISRSGGYTYYADSVKG |
| 11 | CDR1 (Abm) | GSNISSYVMG |
| 12 | CDR1 (Abm) | GSTISSYVMG |
| 13 | CDR2 (Abm) | AISRSGGYTY |
| 14 | CDR3 (Kabat/Abm) | GRYSAWYSQSYEYDY |

Note:
all CDRs are defined according to the Abm convention, unless indicated otherwise.

Generally, the serum albumin-binding ISVD's provided by the present invention are variants of the sequences of SEQ ID NO: 18 and 19, in that:
they have the same CDRs (or essentially the same CDR's) as the sequence of SEQ ID NO:18 or the sequence of SEQ ID NO: 19; and
they have a certain degree of sequence identity with the sequence of SEQ ID NO:18 and/or 19 (which degree of sequence identity is as further described herein).

In particular, serum albumin-binding ISVD's provided by the present invention will generally have a (limited) number of “amino acid differences” (as described herein) compared to the sequence of SEQ ID NO: 18 and/or 19. These amino acid differences may be present in the CDR's (as long as the resulting amino acid sequences as such that they retain the further properties of the amino acid sequences of the invention that are set out herein) and/or be present in the framework regions, and may in particular be present in the framework regions (as defined according to Kabat and/or according to Abm). For example and without limitation, these amino acid differences may for example be humanizing substitutions, substitutions that improve expression in a desired host cell or host organism, substitutions that improve stability and/or resistance to degradation and/or proteases, mutations that reduce binding by pre-existing antibodies, and/or other mutations that are intended to optimize the sequence of the amino acid sequences of the invention; or any suitable combination of such amino acid differences. Reference is made to the further disclosure herein.

In a first aspect, the invention relates to an ISVD that can bind (and in particular, specifically bind) to human serum albumin, and that has:
a CDR1 (according to Kabat) that is the amino acid sequence SYVMG (SEQ ID NO: 9) or an amino acid sequence that has 2 or 1 amino acid difference(s) (as defined herein) with the amino acid sequence of SEQ ID NO: 9; and
a CDR2 (according to Kabat) that is the amino acid sequence AISRSGGYTYYADSVKG (SEQ ID NO:10) or an amino acid sequence that has 3, 2 or 1 amino acid difference(s) (as defined herein) with the amino acid sequence of SEQ ID NO: 10; and
a CDR3 (according to Kabat) that is the amino acid sequence GRYSAWYSQSYEYDY (SEQ ID NO: 14) or an amino acid sequence that has 3, 2 or 1 amino acid difference(s) (as defined herein) with the amino acid sequence of SEQ ID NO: 14.

In particular, a serum albumin binder according to this aspect of the invention may be (and preferably is) as further described herein.

In a more specific aspect, the invention relates to an ISVD that can bind (and in particular, specifically bind) to human serum albumin, and that has:
a CDR1 (according to Kabat) that is the amino acid sequence SYVMG (SEQ ID NO: 9); and
a CDR2 (according to Kabat) that is the amino acid sequence AISRSGGYTYYADSVKG (SEQ ID NO:10); and
a CDR3 (according to Kabat) that is the amino acid sequence GRYSAWYSQSYEYDY (SEQ ID NO: 14).

Again, a serum albumin binder according to this aspect of the invention may be (and preferably is) as further described herein.

In another aspect, the invention relates to an ISVD that can bind (and in particular, specifically bind) to human serum albumin, and that has:
a CDR1 (according to Abm) that is the amino acid sequence GSNISSYVMG (SEQ ID NO:11) or GSTISSYVMG (SEQ ID NO:12) or an amino acid sequence that has 2 or 1 amino acid difference(s) (as defined herein) with the amino acid sequence of SEQ ID NO: 11 or SEQ ID NO: 12; and
a CDR2 (according to Abm) that is the amino acid sequence AISRSGGYTY (SEQ ID NO: 13) or an amino acid sequence that has 3, 2 or 1 amino acid difference(s) (as defined herein) with the amino acid sequence of SEQ ID NO: 13; and
a CDR3 (according to Abm) that is the amino acid sequence GRYSAWYSQSYEYDY (SEQ ID NO: 14) or an amino acid sequence that has 3, 2 or 1 amino acid difference(s) (as defined herein) with the amino acid sequence of SEQ ID NO: 14.

In particular, a serum albumin binder according to this aspect of the invention may be (and preferably is) as further described herein.

In a more specific aspect, the invention relates to an ISVD that can bind (and in particular, specifically bind) to human serum albumin, and that has:
a CDR1 (according to Abm) that is the amino acid sequence GSNISSYVMG (SEQ ID NO:11) or GSTISSYVMG (SEQ ID NO:12); and
a CDR2 (according to Abm) that is the amino acid sequence AISRSGGYTY (SEQ ID NO: 13); and
a CDR3 (according to Abm) that is the amino acid sequence GRYSAWYSQSYEYDY (SEQ ID NO: 14).

Again, a serum albumin binder according to this aspect of the invention may be (and preferably is) as further described herein.

Generally, the serum albumin binders according to the different aspects of the invention are preferably such that they have:
a degree of sequence identity with the sequence of SEQ ID NO: 18 and/or 19 (in which the CDR's and any C-terminal extension that may be present are not taken into account for determining the degree of sequence identity) of at least 85%, preferably at least 90%, more preferably at least 95%;
   and/or such that they have:
and/or have no more than 7, preferably no more than 5, such as only 3, 2 or 1 “amino acid differences” (as defined herein, and not taking into account the CDRs and any C-terminal extension that may be present) with the sequence of SEQ ID NO: 18 and/or 19.

The serum albumin binders according to the different aspects of the invention are generally preferably such that they bind to human serum albumin with a dissociation constant (KD) of 10⁻⁵ to 10⁻¹² moles/liter or less, and preferably 10⁻⁷ to 10⁻¹² moles/liter or less and more preferably 10⁻⁸ to 10⁻¹² moles/liter, and/or with a binding affinity of at least 10⁷ M⁻¹, preferably at least 10⁸ M⁻¹, more preferably at least 10⁹ M⁻¹, such as at least 10¹² M⁻¹, as determined using ProteOn (reference is made to Example 1). Preferably, a serum albumin binder of the invention will bind to the desired antigen with an affinity less than 500 nM, preferably less than 200 nM, more preferably less than 10 nM, such as less than 500 pM, again as determined using ProteOn (reference is again made to Example 1).

The serum albumin binders according to the different aspects of the invention are preferably also such that they compete with the amino acid sequence of SEQ ID NO: 18 for binding to (human) serum albumin and/or that they “cross-block” (as defined herein) the binding of the amino acid sequence of SEQ ID NO:18 to (human) serum albumin.

Preferably, the albumin binders of the invention are such that they bind to essentially the same amino acid residues and/or epitope on human serum albumin as SEQ ID NO:18, and even more preferably such that they share essentially the same amino acid interactions SEQ ID NO:18. For this purpose, according to a specific but non-limiting aspect, the albumin binders of the invention preferably either have the same CDRs as the sequence of SEQ ID NO:18, or compared to the sequence of SEQ ID NO: 18 preferably contain within their CDR's only such mutations (such as conservative amino acid substitutions) that still allow them to undergo the same or essentially the same amino acid interactions with human serum albumin as SEQ ID NO:18.

The serum albumin binders according to the different aspects of the invention are generally preferably also such that they are cross-reactive between human serum albumin and serum albumin from at least one, preferably from at least two, more preferably from at least three and up to essentially all of the following species of mammal: dog, rat, mouse, rabbit, guinea pig, pig, sheep, cow and cynomolgus monkey. In particular, the serum albumin binders according to the different aspects of the invention are such that they are (at least) cross-reactive between human serum albumin and dog serum albumin, and preferably also between either human serum albumin and/or dog serum albumin on the one hand, and at least one, preferably at least two, more preferably at all three of rat serum albumin, mouse serum albumin and serum albumin from cynomolgus monkey on the other hand. In this respect, the serum albumin binders of the invention may have improved cross-reactivity (in particular between human serum albumin on the one hand and dog serum albumin on the other hand) compared to serum albumin binders that have (essentially) the same CDR's as Alb-11 and/or Alb-23D. Reference is made to the data in the Experimental Part below.

For the sake of reference, FIG. 11 gives an alignment of serum albumin from different species of mammal (source: macromoleculeinsights.com/albumin.php, the amino acid numbering in FIG. 11 is the numbering used on said webpage). For the sake of convenience, in the sequence of human serum albumin, the stretches of amino acids that are assumed to be part of the putative epitope of the amino acid sequences of the invention have been highlighted. Without being limited to any specific mechanism or hypothesis, it is assumed that the amino acid sequences of the invention are (essentially) capable of binding to (one or more amino acid residues within) the corresponding stretches of amino acid residues that are present within the amino acid sequence of those mammalian serum albumins that the amino acid sequences of the invention are cross-reactive with.

Generally, a serum albumin binder of the invention can be considered to be cross-reactive between human serum albumin and serum albumin from one of these species when it can bind to human serum albumin with an affinity less than 500 nM, preferably less than 200 nM, more preferably less than 10 nM; and also to the serum albumin from said species with an affinity less than 500 nM, preferably less than 200 nM, more preferably less than 10 nM, again both as determined using ProteOn (reference is again made to Example 1).

The serum albumin binders according to the different aspects of the invention are preferably also such that either:
they have a serum half-life in man (expressed as t1/2 beta) that is more than 6 hours, preferably more than 12 hours, more preferably of more than 24 hours, even more preferably more than 72 hours; for example of about one week, two weeks and up to the half-life of serum albumin in man (estimated to be around 19 days);
   and/or such that:
when it is linked to a therapeutic moiety or entity, it confers to the resulting polypeptide of the invention a serum half-life in man (expressed as t1/2 beta) that is more than 6 hours, preferably more than 12 hours, more preferably of more than 24 hours, even more preferably more than 72 hours; for example of about one week, two weeks and up to the half-life of serum albumin in man (estimated to be around 19 days)

The half-life in mammalian species other than man will, among other factors, mainly depend on the binding properties (such as affinity) of the albumin binder of the invention for the serum albumin from said mammalian species as well on the half-life of the naïve serum albumin in said species. According to a preferred embodiment of the invention, when a serum albumin binder of the invention is cross-reactive (as defined herein) between human serum albumin and serum albumin from another mammalian species, then the half-life of the serum albumin binder of the invention (and/or of a compound of the invention comprising said serum albumin binder) as determined in said species is preferably at least 5%, such as at least 10%, more preferably at least 25%, for example about 50% and possibly up to 100% of the half-life of the naïve serum albumin in said species.

Compared to the sequence of SEQ ID NO: 18, the serum albumin binders of the invention preferably also contain (at least):
one or more humanizing substitutions; and/or
one or more mutations (i.e. amino acid substitutions, deletions or additions, and in particular substitutions) that reduce the binding by pre-existing antibodies; and may optionally contain one or more further mutations as described herein.

For suitable humanizing substitutions (and suitable combinations thereof), reference is for example made to WO 09/138519 (or in the prior art cited in WO 09/138519) and WO 08/020079 (or in the prior art cited in WO 08/020079), as well as Tables A-3 to A-8 from WO 08/020079 (which are lists showing possible humanizing substitutions). Some preferred but non-limiting examples of such humanizing substitutions are Q108L and A14P or a suitable combination thereof. Such humanizing substitutions may also be suitably combined with one or more other mutations as described herein (such as with one or more mutations that reduce binding by pre-existing antibodies).

For suitable mutations that can reduce the binding by pre-existing antibodies (and suitable combinations of such mutations), reference is for example made to WO 2012/175741 and WO 2015/173325 and also to for example WO 2013/024059 and WO 2016/118733. As described therein, such mutations can comprise (a suitable combination of) one or more amino acid substitutions, deletions or additions (and in particular substitutions), which mutations will often be in the so-called C-terminal region of the ISV. For example, such mutations can comprise mutations (and in particular substitutions) at one or more of positions 11, 13, 14, 15, 40, 41, 42, 82, 82a, 82b, 83, 84, 85, 87, 88, 89, 103, 108 and/or mutations at one or more positions in the C-terminal VTVSS sequence (i.e. positions 109, 110, 111, 112 and 113), with one or more mutations at positions 11, 89, 110 and/or 112 being particularly preferred. Some preferred but non-limiting examples of such mutations are suitable substitutions (where required) such that after the mutation, at the indicated position, one of the following amino acid residues is present: 11L, 11K, 11V, 14A, 14P, 41A, 41L, 41P, 41S, 41T, 42E, 42G, 87A, 87T, 89A, 89L, 89T, 108L, 110K, 110Q, 112K and/or 112Q (with 11L, 89A, 89L, 89T, 110K, 110Q, 112K and 112Q being particularly preferred); or any suitable combination of such substitutions, such as for example and without limitation: 11V in combination with 89L or 89T; 11V in combination with 110K or 110Q; or 11V in combination with 89L and 110K or 110Q. Such mutations that reduce binding by pre-existing antibodies may also be suitably combined with one or more other mutations as described herein (such as with one or more humanizing substitutions).

Where appropriate (as further described herein, and in particular when the serum albumin binder of the invention is present at and/or forms the C-terminal end of the compound of the invention in which it is present), for reducing the binding of pre-existing antibodies, the serum albumin binders of the invention (and, as further described herein, also the compounds of invention) may also comprise a C-terminal extension (such as a C-terminal alanine residue). As described in WO 2012/175741, such a C-terminal extension reduces binding by pre-existing antibodies. A suitable C-terminal extension can generally be further described herein and can in particular have the formula —(X)ₙ, in which X can be any naturally occurring amino acid (but preferably not cysteine) and n can be 1, 2, 3, 4 or 5. Reference is again made to WO 2012/175741, to also to for example WO 2015/173325, WO 2013/024059 and WO 2016/118733. The presence of such a C-terminal extension may also be suitably combined with one or more of the other mutations described herein (such as with one or more humanizing substitutions and/or one or more mutations that reduce binding by pre-existing antibodies).

Other mutations that may be present in the serum albumin binders of the invention for example and without limitation include one or more mutations (an in particular substitutions) that improve expression in a desired host cell or host organism, one or more mutations (and in particular substitutions) that improve stability and/or resistance to degradation and/or proteases, and/or one or more other mutations that are intended to optimize the sequence of the amino acid sequences of the invention (for example and without limitation, one or more mutations that (further) reduce any tendency of the albumin binders to form dimers); or any suitable combination of such mutations.

Some non-limiting examples of such mutations are suitable substitutions (where required) such that after the mutation, at the indicated position, one of the following amino acid residues is present: 28T, 39Q, 74S, 76N, 78L and 83R; or any suitable combination of such substitutions (for example so as to form an SKN motif at positions 74-76). Also, where appropriate (as further described herein), the serum albumin binders of the invention may have a D at position 1 (i.e. a EID mutation compared to the sequence of SEQ ID NO:18 and/or 19), in particular when the serum albumin binder of the invention is present at and/or forms the N-terminal end of the compound of the invention in which it is present. Such mutations may again be suitably combined with one or more other mutations as described herein (such as with one or more humanizing substitutions and/or one or more mutations that reduce binding by pre-existing antibodies).

Other mutations that may be present in the amino acid sequences of the invention will be clear to the skilled person based on the disclosure herein.

It is also possible that a single mutation (or a suitable combination of mutations) provides multiple functionalities or advantages. For example and without limitation, a humanizing Q108L substitution may also reduce binding by pre-existing antibodies.

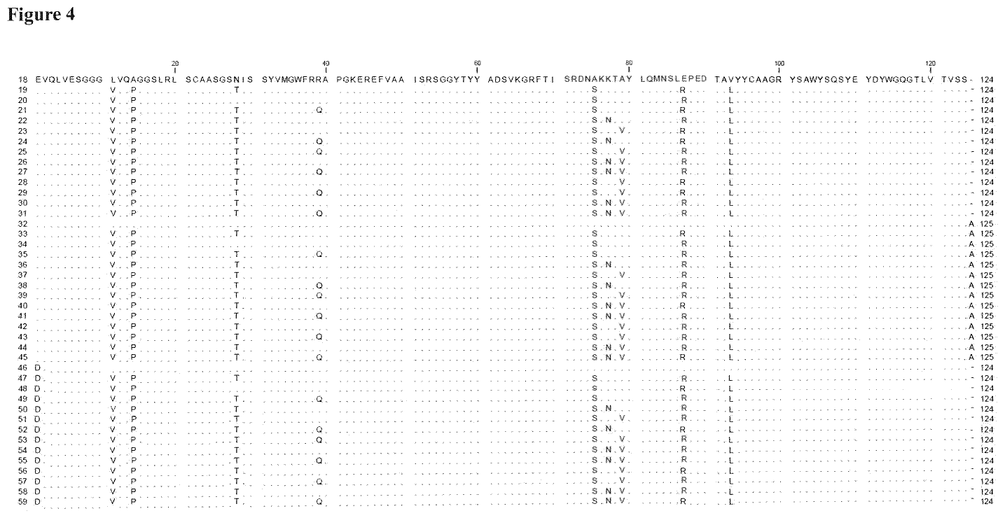

Some preferred but non-limiting examples of amino acid residues (i.e. mutations compared to the amino acid sequence of SEQ ID NO:18) that may be present in the amino acid sequences of the invention (i.e. by themselves or in suitable combination) include: 11V (i.e. L11V), 14P (i.e. A14P), 28T (i.e. N28T), 39Q (i.e. R39Q), 74S (i.e. A74S), 76N (i.e. K76N), 78L (i.e. A78L), 83R (i.e. E83R), 89L (i.e. V89L), 89T (i.e. V89T), 110K (e.g. T110K) or 110Q (e.g. T110Q); as well as, where appropriate (as further described herein), 1D (e.g. E1D) and/or a C-terminal extension (X)ₙ as defined herein (such as 114A). Reference is also made to sequences and mutations shown in FIGS. 4A and 4B. For example, some preferred but non-limiting examples of suitable combinations of such amino acid residues (i.e. mutations compared to the amino acid sequence of SEQ ID NO:18) include:
L11V,A14P,N28T,R39Q,A74S,E83R,V89L;
L11V,A14P,N28T,A74S,K76N,E83R,V89L;
L11V,A14P,N28T,A74S,A78V,E83R,V89L;
L11V,A14P,N28T,R39Q,A74S,K76N,E83R, V89L;
L11V,A14P,N28T,R39Q,A74S,K76N,E83R,V89L;
L11V,A14P,N28T,R39Q,A74S,A78V,E83R,V89L;
L11V,A14P,N28T,A74S,K76N,A78V,E83R,V89L;
L11V,A14P,N28T,R39Q,A74S,K76N,A78V,E83R,V89L;
L11V,A14P,N28T,A74S,A78L,E83R,V89L;
L11V,A14P,N28T,R39Q,A74S,A78L,E83R,V89L;
L11V,A14P,N28T,A74S,K76N,A78L,E83R, V89L; and
L11V,A14P,N28T,R39Q,A74S,K76N,A78L,E83R,V89L;
   and other suitable combinations will be clear to the skilled person based on the disclosure herein.

Some preferred but non-limiting amino acid sequences of the invention are those in which (i) position 11 is V and position 89 is L; (ii) position 89 is T; (iii) positions 74-76 form an SKN motif; (iv) position 11 is V, position 89 is L and positions 74-76 form an SKN motif;

and (iv) position 89 is T and positions 74-76 form an SKN motif; which amino acid sequences of the invention are as further described herein (and accordingly can also suitably contain one or more further amino acid mutations as described herein).

Some preferred, but non-limiting examples of the amino acid sequences of the invention are:
SEQ ID NOs: 18 to 31, which are examples of amino acid sequences of the invention without a C-terminal alanine extension;
SEQ ID NOs: 32 to 45, which are examples of amino acid sequences of the invention with a C-terminal extension (in each case, exemplified by means of a C-terminal alanine extension, which is generally the preferred C-terminal extension); and
SEQ ID NOs: 46 to 59, which are examples of amino acid sequences of the invention with an N-terminal E1D mutation).

Based on the further disclosure herein, it will be clear to the skilled person that in practice:
albumin binders of the invention with a C-terminal extension (such as those of SEQ ID NOs: 32 to 45) will often be used as/present at the C-terminal end of the polypeptides of the invention (as defined herein) in which they are present;
albumin binders of the invention with an EID mutation (such as those of SEQ ID NOs: 46 to 59) will often be used as/present at the N-terminal end of the polypeptides of the invention in which they are present;
albumin binders of the invention without a C-terminal extension and without an EID mutation (such as those of SEQ ID NOs: 18 to 31) will often be present somewhere in the “middle” of a polypeptide of the invention.

Each of the amino acid sequences of SEQ ID NOs: 18 to 59, as well as proteins, polypeptides and other compounds and constructs comprising the same (as further described herein), form further aspects of the present invention.

In a further aspect, the invention relates to an amino acid sequence which is one of the amino acid sequences of SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58 or SEQ ID NO:59; and each of these amino acid sequences of the invention (as well as polypeptides of the invention
as defined herein—that comprise such an amino acid sequence of the invention) forms a further aspect of the present invention.

As further described herein, the amino acid sequences provided by the invention are proteins that can bind to, and that can in particular specifically (as described herein) bind to, human serum albumin. Thus, they can be used as binding units or binding domains for binding to (human) serum albumin, for example to confer an increase in half-life (as defined herein) to therapeutic compounds, moieties or entities. For the use of serum albumin-binding domains to increase half-life of therapeutic compounds, moieties or entities, reference is for example made to WO 2004/041865, WO 2006/122787, EP 2 139 918, WO 2011/006915, WO 2012/175400 and/or WO 2014/111550. The serum albumin binders of the invention can generally be used in the same way and for the same purposes as the serum albumin binders described in these references.

In some further non-limiting aspects, the invention also relates to:
proteins, polypeptides and other constructs, molecules or chemical entities that comprise or essentially consist of at least one serum albumin binder of the invention as described herein (again, also referred to herein as “compounds of the invention” or as “polypeptides of the invention”);
methods for expressing/producing a serum albumin binder of the invention and/or a compound of the invention;
a host cell, host organism or other (expression) system that can express or produce a serum albumin binder of the invention and/or a compound of the invention;

compositions and products (such as pharmaceutical compositions and products) that comprise a serum albumin binder of the invention and/or a compound of the invention;
nucleotide sequences and nucleic acids, such as (expression) vectors, that encode a serum albumin binder of the invention and/or a compounds of the invention;
uses of the compounds of the invention and/or the compounds of the invention, such as the use of a compound of the invention to increase the (serum) half-life of a therapeutic compounds, moiety or entity and the therapeutic and/or prophylactic use of a compound of the invention.

These and further aspects, embodiments, advantages, applications and uses of the invention will become clear from the further description herein.

In the present specification:
the term “immunoglobulin single variable domain” (also referred to as “ISV” or “ISVD”) is generally used to refer to immunoglobulin variable domains (which may be heavy chain or light chain domains, including VH, VHH or VL domains) that can form a functional antigen binding site without interaction with another variable domain (e.g. without a VH/VL interaction as is required between the VH and VL domains of conventional 4-chain monoclonal antibody). Examples of ISVDs will be clear to the skilled person and for example include Nanobodies (including a VHH, a humanized VHH and/or a camelized VHs such as camelized human VHs), IgNAR, domains, (single domain) antibodies (such as dAbs™) that are VH domains or that are derived from a VH domain and (single domain) antibodies (such as dAbs™) that are VL domains or that are derived from a VL domain. Unless explicitly mentioned otherwise herein, ISVDs that are based on and/or derived from heavy chain variable domains (such as VH or VHH domains) are generally preferred. Most preferably, unless explicitly indicated otherwise herein, an ISVD will be a Nanobody.
the term “Nanobody” is generally as defined in WO 2008/020079 or WO 2009/138519, and thus in a specific aspect generally denotes a VHH, a humanized VHH or a camelized VH (such as a camelized human VH) or generally a sequence optimized VHH (such as e.g. optimized for chemical stability and/or solubility, maximum overlap with known human framework regions and maximum expression). It is noted that the terms Nanobody or Nanobodies are registered trademarks of Ablynx N.V. and thus may also be referred to as Nanobody® and/or Nanobodies®);
Generally, unless indicated otherwise herein, the ISVD's, Nanobodies, polypeptides, proteins and other compounds and constructs referred to herein will be intended for use in prophylaxis or treatment of diseases or disorders in man (and/or optionally also in warm-blooded animals and in particular mammals). Thus, generally, the ISVD's, Nanobodies, polypeptides, proteins and other compounds and constructs described herein are preferably such that they can be used as, and/or can suitably be a part of, a (biological) drug or other pharmaceutically or therapeutically active compound and/or of a pharmaceutical product or composition. Such a drug, compound or product is preferably such that it is suitable for administration to a human being, e.g. for prophylaxis or treatment of a subject in need of such prophylaxis or treatment or for example as part of a clinical trial. As further described herein, for this purpose, such a drug or compound may contain other moieties, entities or binding units besides the ISVDs provided by the invention (which, as also described herein, may for example be one or more other further therapeutic moieties and/or one or more other moieties that influence the pharmacokinetic or pharmacodynamic properties of the ISVD-based or Nanobody-based biological, such as its half-life). Suitable examples of such further therapeutic or other moieties will be clear to the skilled person, and for example generally can include any therapeutically active protein, polypeptide or other binding domain or binding unit, as well as for example modifications such as those described on pages 149 to 152 of WO 2009/138159. An ISVD-based biological or Nanobody-based biological is preferably a therapeutic or intended for use as a therapeutic (which includes prophylaxis and diagnosis) and for this purpose preferably contains at least one ISVD against a therapeutically relevant target (such as for example RANK-L, vWF, IgE, RSV, CXCR4, IL-23 or other interleukins, etc.). For some specific but non-limiting examples of such ISVD-based or Nanobody-based biologicals, reference is to Examples 8 to 18 and also for example made to the various applications by Ablynx N.V. (such as for example and without limitation WO 2004/062551, WO 2006/122825, WO 2008/020079 and WO 2009/068627), as well as for example (and without limitation) to applications such as WO 2006/038027, WO 2006/059108, WO 2007/063308, WO 2007/063311, WO 2007/066016 and WO 2007/085814. Also, as further described herein, the further moiety may be an ISVD or Nanobody as described herein directed against a (human) serum protein such as (human) serum albumin, and such an ISVD or Nanobody may also find therapeutic uses, in particular in and/or for extending the half-life of the TNF binders described herein. Reference is for example made to WO 2004/041865, WO 2006/122787 and WO 2012/175400, which generally describe the use of serum-albumin binding Nanobodies for half-life extension. Also, in the present specification, unless explicitly mentioned otherwise herein, all terms mentioned herein have the meaning given in WO 2009/138519 (or in the prior art cited in WO 2009/138519) or WO 2008/020079 (or in the prior art cited in WO 2008/020079). Also, where a method or technique is not specifically described herein, it can be performed as described in WO 2009/138519 (or in the prior art cited in WO 2009/138519) or WO 2008/020079 (or in the prior art cited in WO 2008/020079). Also, as described herein, any pharmaceutical product or composition comprising any ISVD or compound of the invention may also comprise one or more further components known per se for use in pharmaceutical products or compositions (i.e. depending on the intended pharmaceutical form) and/or for example one or more other compounds or active principles intended for therapeutic use (i.e. to provide a combination product).

Also, when used in the present specification or claims, the following terms have the same meaning as given on, and/or where applicable can be determined in the manner described in, pages 62-75 of WO 2009/138519: “agonist”, “antagonist”, “inverse agonist”, “non-polar, uncharged amino acid residue”, “polar uncharged amino acid residue”, “polar, charged amino acid residue”, “sequence identity”, “exactly the same” and “amino acid difference” (when referring to a sequence comparison of two amino acid sequences), “(in) essentially isolated (form)”, “domain”, “binding domain”, “antigenic determinant”, “epitope”, “against” or “directed against” (an antigen), “specificity” and “half-life”. In addition, the terms “modulating” and “to modulate”, “interaction site”, “specific for”, “cross-block”, “cross-blocked” and “cross-blocking” and “essentially independent of the pH” are as defined on (and/or can be determined as described on) pages 74-79 of WO 2010/130832 of Ablynx N.V. Also, when referring to a construct, compound, protein or polypeptide of the invention, terms like “monovalent”, “bivalent” (or “multivalent”), “bispecific” (or “multispecific”), and “biparatopic” (or “multiparatopic”) may have the meaning given in WO 2009/138519, WO 2010/130832 or WO 2008/020079.

The term “half-life” as used herein in relation to an ISVD, Nanobody, ISVD-based biological, Nanobody-based biological or any other amino acid sequence, compound or polypeptide referred to herein can generally be defined as described in paragraph o) on page 57 of WO 2008/020079 and as mentioned therein refers to the time taken for the serum concentration of the amino acid sequence, compound or polypeptide to be reduced by 50%, in vivo, for example due to degradation of the sequence or compound and/or clearance or sequestration of the sequence or compound by natural mechanisms. The in vivo half-life of an amino acid sequence, compound or polypeptide of the invention can be determined in any manner known per se, such as by pharmacokinetic analysis. Suitable techniques will be clear to the person skilled in the art, and may for example generally be as described in paragraph o) on page 57 of WO 2008/020079. As also mentioned in paragraph o) on page 57 of WO 2008/020079, the half-life can be expressed using parameters such as the t1/2-alpha, t1/2-beta and the area under the curve (AUC). In this respect it should be noted that the term “half-life” as used herein in particular refers to the t1/2-beta or terminal half-life (in which the t1/2-alpha and/or the AUC or both may be kept out of considerations). Reference is for example made to the Experimental Part below, as well as to the standard handbooks, such as Kenneth, A et al: Chemical Stability of Pharmaceuticals: A Handbook for Pharmacists and Peters et al, Pharmacokinetic analysis: A Practical Approach (1996). Reference is also made to “Pharmacokinetics”, M Gibaldi & D Perron, published by Marcel Dekker, 2nd Rev. edition (1982). Similarly, the terms “increase in half-life” or “increased half-life” are also as defined in paragraph o) on page 57 of WO 2008/020079 and in particular refer to an increase in the t1/2-beta, either with or without an increase in the t1/2-alpha and/or the AUC or both.

When a term is not specifically defined herein, it has its usual meaning in the art, which will be clear to the skilled person. Reference is for example made to the standard handbooks, such as Sambrook et al, “Molecular Cloning: A Laboratory Manual” (2nd.Ed.), Vols. 1-3, Cold Spring Harbor Laboratory Press (1989); F. Ausubel et al, eds., “Current protocols in molecular biology”, Green Publishing and Wiley Interscience, New York (1987); Lewin, “Genes II”, John Wiley & Sons, New York, N.Y., (1985); Old et al., “Principles of Gene Manipulation: An Introduction to Genetic Engineering”, 2nd edition, University of California Press, Berkeley, CA (1981); Roitt et al., “Immunology” (6th. Ed.), Mosby/Elsevier, Edinburgh (2001); Roitt et al., Roitt's Essential Immunology, 10th Ed. Blackwell Publishing, UK (2001); and Janeway et al., “Immunobiology” (6th Ed.), Garland Science Publishing/Churchill Livingstone, New York (2005), as well as to the general background art cited herein.

Also, as already indicated herein, the amino acid residues of a Nanobody are numbered according to the general numbering for VHs given by Kabat et al. (“Sequence of proteins of immunological interest”, US Public Health Services, NIH Bethesda, MD, Publication No. 91), as applied to VHH domains from Camelids in the article of Riechmann and Muyldermans, J. Immunol. Methods 2000 Jun. 23; 240 (1-2): 185-195; or referred to herein. According to this numbering, FR1 of a Nanobody comprises the amino acid residues at positions 1-30, CDR1 of a Nanobody comprises the amino acid residues at positions 31-35, FR2 of a Nanobody comprises the amino acids at positions 36-49, CDR2 of a Nanobody comprises the amino acid residues at positions 50-65, FR3 of a Nanobody comprises the amino acid residues at positions 66-94, CDR3 of a Nanobody comprises the amino acid residues at positions 95-102, and FR4 of a Nanobody comprises the amino acid residues at positions 103-113. [In this respect, it should be noted that—as is well known in the art for VH domains and for VHH domains—the total number of amino acid residues in each of the CDR's may vary and may not correspond to the total number of amino acid residues indicated by the Kabat numbering (that is, one or more positions according to the Kabat numbering may not be occupied in the actual sequence, or the actual sequence may contain more amino acid residues than the number allowed for by the Kabat numbering). This means that, generally, the numbering according to Kabat may or may not correspond to the actual numbering of the amino acid residues in the actual sequence. Generally, however, it can be said that, according to the numbering of Kabat and irrespective of the number of amino acid residues in the CDR's, position 1 according to the Kabat numbering corresponds to the start of FR1 and vice versa, position 36 according to the Kabat numbering corresponds to the start of FR2 and vice versa, position 66 according to the Kabat numbering corresponds to the start of FR3 and vice versa, and position 103 according to the Kabat numbering corresponds to the start of FR4 and vice versa.].

Alternative methods for numbering the amino acid residues of VH domains, which methods can also be applied in an analogous manner to VHH domains from Camelids and to Nanobodies, are the method described by Chothia et al. (Nature 342, 877-883 (1989)), the so-called “AbM definition” and the so-called “contact definition”. However, in the present description, aspects and figures, the numbering according to Kabat as applied to VHH domains by Riechmann and Muyldermans will be followed, unless indicated otherwise.

It should also be noted that the Figures, any Sequence Listing and the Experimental Part/Examples are only given to further illustrate the invention and should not be interpreted or construed as limiting the scope of the invention and/or of the appended claims in any way, unless explicitly indicated otherwise herein.

As further described herein, the serum albumin binders of the invention can be used with advantage as a moiety, binding unit or fusion partner in order to increase the half-life of therapeutic compounds, moieties or entities such as polypeptides, proteins, compounds (including, without limitation, small molecules) or other therapeutic entities.

Thus, in another aspect, the invention provides polypeptides, proteins, constructs, compounds or other chemical entities that comprise or essentially consist of a serum albumin binder of the invention and one or more other amino acid sequences, (binding) domains, binding units or other moieties or chemical entities.

In particular, the invention provides polypeptides, proteins, constructs, compounds or other chemical entities that comprise a serum albumin binder of the invention and one or more (such as one or two) therapeutic moieties (which may be the same or different, and may for example be directed against the same target or to different targets, and when they are directed to the same target may be directed towards the same or different epitopes, parts, domains or subunits of said target), suitably linked to each other either directly or via one or more suitable linkers or spacers. Such polypeptides, proteins or constructs may for example and without limitation be a fusion protein, as further described herein.

The invention further relates to therapeutic uses of such polypeptides, proteins, constructs or compounds and to pharmaceutical compositions comprising such polypeptides, proteins, constructs or compounds.

In one aspect, the at least one therapeutic moiety comprises or essentially consists of a therapeutic protein, polypeptide, compound, factor or other entity. In a preferred embodiment the therapeutic moiety is directed against a desired antigen or target, is capable of binding to a desired antigen (and in particular capable of specifically binding to a desired antigen), and/or is capable of interacting with a desired target. In another embodiment, the at least one therapeutic moiety comprises or essentially consists of a therapeutic protein or polypeptide. In a further embodiment, the at least one therapeutic moiety comprises or essentially consists of a binding domain or binding unit, such as an immunoglobulin or immunoglobulin sequence (including but not limited to a fragment of an immunoglobulin), such as an antibody or an antibody fragment (including but not limited to an ScFv fragment), or of another suitable protein scaffold, such as protein A domains (such as Affibodies™), tendamistat, fibronectin, lipocalin, CTLA-4, T-cell receptors, designed ankyrin repeats, avimers and PDZ domains (Binz et al., Nat. Biotech 2005, Vol 23:1257), and binding moieties based on DNA or RNA including but not limited to DNA or RNA aptamers (Ulrich et al., Comb Chem High Throughput Screen 2006 9(8):619-32).

In yet another aspect, the at least one therapeutic moiety comprises or essentially consists of an antibody variable domain, such as a heavy chain variable domain or a light chain variable domain.

In a preferred aspect, the at least one therapeutic moiety comprises or essentially consists of at least one immunoglobulin single variable domain, such as a domain antibody, single domain antibody, “dAb” or Nanobody (such as a VHH, a humanized VHH or a camelized VH) or an IgNAR domain.

In a specific embodiment, the at least one therapeutic moiety comprises or essentially consists of at least one monovalent Nanobody or a bivalent, multivalent, bispecific or multispecific Nanobody construct.

The polypeptides, (fusion) proteins, constructs or compounds that comprise a serum albumin binder of the invention and one or more therapeutic moieties can generally be (prepared and used) as described in the prior art cited above (such as WO 04/041865, WO 06/122787, WO 2012/175400 and WO 2015/173325; reference is also made to for example, WO 2004/003019, EP 2 139 918, WO 2011/006915 and WO 2014/111550) with a serum albumin binder of the invention instead of the half-life increasing moieties described in said

The polypeptides, (fusion) proteins, constructs or compounds that comprise a serum albumin binder of the invention and one or more therapeutic moieties will generally and preferably have an increased half-life (as described herein, and preferably expressed as t1/2-beta), compared to the therapeutic moiety or moieties per se.

Generally, the compounds, polypeptides, constructs or fusion proteins described herein preferably have a half-life (again, as described herein, and preferably expressed as t1/2-beta) that is at least 1.5 times, preferably at least 2 times, such as at least 5 times, for example at least 10 times or more than 20 times, greater than the half-life of the corresponding therapeutic moiety per se (as measured in either in man or a suitable animal, such as mouse or cynomolgus monkey).

Also, preferably, any such compound, polypeptide, fusion protein or construct has a half-life (again, as described herein, and preferably expressed as t1/2-beta) in man that is increased with more than 1 hour, preferably more than 2 hours, more preferably of more than 6 hours, such as of more than 12 hours, compared to the half-life of the corresponding therapeutic moiety per se.

Also, preferably, a compound or polypeptide of the invention has a half-life (again, as described herein, and preferably expressed as t1/2-beta) in man that is more than 1 hour, preferably more than 2 hours, more preferably of more than 6 hours, such as of more than 12 hours, and for example of about one day, two days, one week, two weeks and up to the half-life of serum albumin in man (estimated to be around 19 days).

As mentioned, in one aspect, a serum albumin binder of the invention is used to increase the half-life of (one or more) immunoglobulin single variable domains, such as domain antibodies, single domain antibodies, “dAb's”, VHH's or Nanobodies (such as VHH's, humanized VHH's or camelized VH's such as camelized human VH's).

Thus, one embodiment of the invention relates to a polypeptide, construct or fusion protein that comprises a serum albumin binder of the invention and one or more (such as one or two) immunoglobulin single variable domain sequences, which are suitably linked to each other, cither directly or optionally via one or more suitable linkers or spacers. As mentioned herein, each such immunoglobulin single variable domain present in such a polypeptide, construct or fusion protein may independently be a domain antibody, single domain antibody, “dAb”' or Nanobody (such as a VHH, humanized VHH or camelized VH, such as a camelized human VH); and according to one specific but non-limiting aspect, at least one (and up to all) of these immunoglobulin single variable domains comprises two or three disulphide bridges. Preferably, all ISVDs present in such a compound of the invention are Nanobodies.

When a compound of the invention has an ISVD at its C-terminal end (such as a serum albumin binder of the invention or an ISVD that is directed against a therapeutic target), then said C-terminal ISVD (and thus, by extension, the entire compound of the invention) preferably has a C-terminal extension at its C-terminal end. This C-terminal extension will be directly linked to the last C-terminal amino acid residue of the ISVD, which will usually be the amino acid residue at position 113 according to Kabat (unless the ISVD contains one or more amino acid deletions such that the sequence of the ISVD ends before position 113). Thus, generally, the C-terminal extension will be directly linked to the C-terminal VTVSS sequence (SEQ ID NO:15) of the C-terminal ISV (and thus, by extension, to the C-terminal VTVSS sequence of the compound of the invention) or the C-terminal sequence of the C-terminal ISVD that corresponds to the C-terminal ISVD sequence (for example, where said C-terminal sequence of the C-terminal ISVD contains one or more substitutions or deletions compared to the usual VTVSS sequence, such as T110K, T110Q, S112K or S112K).

It will also be clear to the skilled person in the case where a compound of the invention has a serum albumin binder of the invention at its C-terminal end, that then said serum albumin binder of the invention will carry said C-terminal extension.

Generally, any C-terminal extension that is used herein (i.e. at the C-terminal end of a compound of the invention and/or at the C-terminal end of a serum albumin binder of the invention) can generally be as described in WO 2012/174741 or WO 2015/173325 (reference is also made to for example WO 2103/024059 and WO2016/118733). In particular, a C-terminal extension may have the formula (X)ₙ, in which n is 1 to 10, preferably 1 to 5, such as 1, 2, 3, 4 or 5 (and preferably 1 or 2, such as 1); and each X is an (preferably naturally occurring) amino acid residue that is independently chosen from naturally occurring amino acid residues (although according to preferred one aspect, it does not comprise any cysteine residues), and preferably independently chosen from the group consisting of alanine (A), glycine (G), valine (V), leucine (L) or isoleucine (I).

According to some preferred, but non-limiting aspects of such C-terminal extensions X₍ₙ₎, X and n can be as follows:

(a) n=1 and X=Ala;
(b) n=2 and each X=Ala;
(c) n=3 and each =Ala;
(d) n=2 and at least one X=Ala (with the remaining amino acid residue(s) X being independently chosen from any naturally occurring amino acid but preferably being independently chosen from Val, Leu and/or Ile);
(e) n=3 and at least one X=Ala (with the remaining amino acid residue(s) X being independently chosen from any naturally occurring amino acid but preferably being independently chosen from Val, Leu and/or Ile);
(f) n=3 and at least two X=Ala (with the remaining amino acid residue(s) X being independently chosen from any naturally occurring amino acid but preferably being independently chosen from Val, Leu and/or Ile);
(g) n=1 and X=Gly;
(h) n=2 and each X=Gly;
(i) n=3 and each X=Gly;
(j) n=2 and at least one X=Gly (with the remaining amino acid residue(s) X being independently chosen from any naturally occurring amino acid but preferably being independently chosen from Val, Leu and/or Ile);
(k) n=3 and at least one X=Gly (with the remaining amino acid residue(s) X being independently chosen from any naturally occurring amino acid but preferably being independently chosen from Val, Leu and/or Ile);
(l) n=3 and at least two X=Gly (with the remaining amino acid residue(s) X being independently chosen from any naturally occurring amino acid but preferably being independently chosen from Val, Leu and/or Ile);
(m) n=2 and each X=Ala or Gly;
(n) n=3 and each X=Ala or Gly;
(o) n=3 and at least one X=Ala or Gly (with the remaining amino acid residue(s) X being independently chosen from any naturally occurring amino acid but preferably being independently chosen from Val, Leu and/or Ile); or
(p) n=3 and at least two X=Ala or Gly (with the remaining amino acid residue(s) X being independently chosen from any naturally occurring amino acid but preferably being independently chosen from Val, Leu and/or Ile);
with aspects (a), (b), (c), (g), (h), (i), (m) and (n) being particularly preferred, with aspects in which n=1 or 2 being preferred and aspects in which n=1 being particularly preferred.

It should also be noted that, preferably, any C-terminal extension present in a serum albumin binder of the invention does not contain a (free) cysteine residue (unless said cysteine residue is used or intended for further functionalization, for example for pegylation).

Some specific, but non-limiting examples of useful C-terminal extensions are the following amino acid sequences: A, AA, AAA, G, GG, GGG, AG, GA, AAG, AGG, AGA, GGA, GAA or GAG.

Preferably also, when a compound of the invention has an ISVD at its C-terminal end (such as a serum albumin binder of the invention or an ISVD that is directed against a therapeutic target), then (at least) said C-terminal ISVD preferably contains, even more preferably in addition to a C-terminal extension as described herein, one or more mutations that reduce binding by pre-existing antibodies (i.e. as described herein for the serum albumin binders of the invention and as more generally described in WO 2012/175741 and WO 2015/173325 and also for example in WO 2013/024059 and WO 2016/118733). In this respect, it will be clear to the skilled person in the case where a compound of the invention has a serum albumin binder of the invention at its C-terminal end, that then (at least) said serum albumin binder of the invention preferably will contain such mutations (i.e. preferably in addition to a C-terminal extension).

More generally, according to a specific aspect of the invention, when a compound of the invention contains two or more ISVDs (e.g. a serum albumin binder of the invention and one or more ISVDs against a therapeutic target), then preferably all these ISVDs contain mutations that reduce binding to pre-existing antibodies (again, preferably in addition to the C-terminal extension that is linked to the C-terminal ISVD if the compound of the invention has an ISVD at its C-terminal end).

When a compound of the invention has an ISVD at its N-terminal end (such as a serum albumin binder of the invention or an ISVD that is directed against a therapeutic target), then said N-terminal ISVD (and thus, by extension, the entire compound of the invention) preferably contain a D at position 1. In this respect, it will again be clear to the skilled person in the case where a compound of the invention has a serum albumin binder of the invention at its N-terminal end, that then said serum albumin binder of the invention will preferably have a D at position 1 (e.g. an E1D mutation compared to for example the sequence of SEQ ID NO: 18 and/or 19, such as the in the amino acid sequences of the invention of SEQ ID NOs: 46 to 59).

In some further aspects, the invention relates to a protein, polypeptide or other compound or construct that comprises or essentially consists of at least one (and preferably only one) serum albumin binder of the invention and at least one (such as one, two or three) therapeutic moiety or entity (in which said serum albumin binder and the one or more therapeutic moieties or entities are suitably linked, optionally via one or more suitable linkers), which protein, polypeptide, compound, construct is such that:
when it has an ISVD at its C-terminal end, then (the C-terminal ISVD of) said protein, polypeptide, compound, construct has a C-terminal extension (X)ₙ (as further described herein) at its C-terminal end; and/or
when it has an ISVD at its C-terminal end, then at least said the C-terminal ISVD contains one or more mutations that reduce the binding of pre-existing antibodies (as further described herein);
when it has an ISVD at its N-terminal end, then (the N-terminal ISVD of) said protein, polypeptide, compound, construct preferably contains a D at position 1; and/or
in which said ISVDs which protein, polypeptide or other compound may also have ISVD at its N-terminal end, in which case said N-terminal ISVD end preferably has a D or an E1D at position 1;
preferably, essentially all of the ISVDs present in said protein, polypeptide, compound, construct contain one or more mutations that reduce the binding of pre-existing antibodies (as further described herein).

According to one specific aspect of the invention, all therapeutic moieties present in a compound of the invention are ISVD's (i.e. ISVDs against a therapeutic target), and in particular heavy-chain ISVDs, and more in particular Nanobodies (i.e. Nanobodies against a therapeutic target).

For example and without limitation, such compounds of the invention may comprise:
one copy of a serum albumin binder of the invention and one ISVD (and preferably Nanobody) against a therapeutic target; or
one copy of a serum albumin binder of the invention and two ISVDs (and preferably two Nanobodies) against a therapeutic target (which ISVDs may be the same or different and when different may be directed against the same target, against different epitopes on the same target or against different therapeutic targets); or
one copy of a serum albumin binder of the invention and three ISVDs (and preferably three Nanobodies) against a therapeutic target (which ISVDs may be the same or different and when different may be directed against the same target, against different epitopes on the same target or against different therapeutic targets).

Some non-limiting examples of constructs, fusion proteins or polypeptides of the invention can be schematically represented as follows, in which “[Alb]” represents a serum albumin binder of the invention, “[therapeutic moiety 1]” and “[therapeutic moiety 2]” represent the therapeutic moieties (which as mentioned may each independently be an immunoglobulin single variable domain), “-” represents a suitable linker (which is optional; suitable examples are 9GS and 35GS linkers) and the N-terminus is on the left hand side and the C-terminus is on the right hand side:

[Alb]−[therapeutic moiety 1][therapeutic moiety 1]−[Alb]−X₍ₙ₎[Alb]−[therapeutic moiety 1]−[therapeutic moiety 1][therapeutic moiety 1]−[therapeutic moiety 1]−[Alb]−X₍ₙ₎[therapeutic moiety 1]−[Alb]−[therapeutic moiety 1][Alb]−[therapeutic moiety 1]−[therapeutic moiety 2][therapeutic moiety 1]−[therapeutic moiety 2]−[Alb]−X₍ₙ₎[therapeutic moiety 1]−[Alb]−[therapeutic moiety 2]

When the therapeutic moieties are ISVDs (and preferably Nanobodies) against a therapeutic target, preferred but non-limiting constructs, fusion proteins or polypeptides of the invention can be schematically represented as follows, in which “[Alb]” represents a serum albumin binder of the invention, “[therapeutic ISVD 1]” and “[therapeutic ISVD 2]” represent ISVDs against a therapeutic target (which ISVDs may be the same or different and when different may be directed against the same target, against different epitopes on the same target or against different therapeutic targets), “-” represents a suitable linker (which is optional), X(n) represents a C-terminal extension as described herein, and the N-terminus is on the left hand side and the C-terminus is on the right hand side:

[Alb]−[therapeutic ISVD 1]−X₍ₙ₎[therapeutic ISVD 1]−[Alb]−X₍ₙ₎[Alb]−[therapeutic ISVD 1]−[therapeutic ISVD 1]−X₍ₙ₎[therapeutic ISVD 1]−[therapeutic ISVD 1]−[Alb]−X₍ₙ₎[therapeutic ISVD 1]−[Alb]−[therapeutic ISVD 1]−X₍ₙ₎[Alb]−[therapeutic ISVD 1]−[therapeutic ISVD 2]−X₍ₙ₎[therapeutic ISVD 1]−[therapeutic ISVD 2]−[Alb]−X₍ₙ₎[therapeutic ISVD 1]−[Alb]−[therapeutic ISVD 2]−X₍ₙ₎

Thus, in another aspect, the invention relates to a multispecific (and in particular bispecific) Nanobody construct that comprises a serum albumin binder of the invention and at least one other Nanobody (such as one or two other Nanobodies, which may be the same or different), in which said at least one other Nanobody is preferably directed against a desired target (which is preferably a therapeutic target) and/or another Nanobody that useful or suitable for therapeutic, prophylactic and/or diagnostic purposes. Again, the serum albumin binder of the invention and the other Nanobodies may be suitably linked to each other either directly or optionally via one or more suitable linkers or spacers.

For a general description of multivalent and multispecific polypeptides containing one or more Nanobodies and their preparation, reference is also made to Conrath et al., J. Biol. Chem., Vol. 276, 10. 7346-7350, 2001; Muyldermans, Reviews in Molecular Biotechnology 74 (2001), 277-302; as well as to for example WO 96/34103, WO 99/23221, WO 04/041862, WO 2006/122786, WO 2008/020079, WO 2008/142164 or WO 2009/068627.

By means of illustration, some examples of compounds of the invention are given in SEQ ID NOS:61 to 67, using the anti-HER2-Nanobody of SEQ ID NO: 60 as a representative example of an anti-target Nanobody, and with the constituent Nanobodies being in different positions in the compound of the invention. The compounds of SEQ ID NOs: 61 to 64 are examples illustrating bivalent bispecific compounds of the invention and the compounds of SEQ ID NOs: 65 to 67 are examples illustrating trivalent bispecific compounds of the invention. In each case, the compounds contain an EID mutation and a C-terminal alanine residue, and contain representative but non-limiting examples of the use of suitable linkers (i.e. a 15GS linker in SEQ ID NOs:62 and 63 and or 35GS linkers in SEQ ID NOs: 61 and 64 to 67).

Some other examples of some specific multispecific and/or multivalent polypeptide of the invention can be found in the applications by Ablynx N.V. mentioned herein. In particular, for a general description of multivalent and multispecific constructs comprising at least one Nanobody against a serum protein for increasing the half-life, of nucleic acids encoding the same, of compositions comprising the same, of the preparation of the aforementioned, and of uses of the aforementioned, reference is made to the International applications WO 04/041865 and WO 06/122787 mentioned above (the serum albumin binders of the invention described herein can generally be used analogously to the half-life extending Nanobodies described therein such as Alb-8), as well as to the general description and specific examples of such constructs given in for example WO 04/041862, WO 2006/122786, WO 2008/020079, WO 2008/142164 or WO 2009/068627.

The invention also relates to nucleotide sequences or nucleic acids that encode the albumin binders, compounds or polypeptides of the invention. The invention further includes genetic constructs that include the foregoing nucleotide sequences or nucleic acids and one or more elements for genetic constructs known per se. The genetic construct may be in the form of a plasmid or vector. Again, such constructs can be generally as described in the published patent applications of Ablynx N.V., such as for example WO 04/041862, WO 2006/122786, WO 2008/020079, WO 2008/142164 or WO 2009/068627.

The invention also relates to hosts or host cells that contain such nucleotide sequences or nucleic acids, and/or that express (or are capable of expressing), the albumin binders, compounds or polypeptides of the invention. Again, such host cells can be generally as described in the published patent applications of Ablynx N.V., such as for example WO 04/041862, WO 2006/122786, WO 2008/020079, WO 2008/142164 or WO 2009/068627.

The invention also relates to a method for preparing an albumin binder, compound or polypeptide of the invention, which method comprises cultivating or maintaining a host cell as described herein under conditions such that said host cell produces or expresses an albumin binder, compound or polypeptide of the invention, and optionally further comprises isolating the albumin binder, compound or polypeptide of the invention so produced. Again, such methods can be performed as generally described in the published patent applications of Ablynx N.V., such as for example WO 04/041862, WO 2006/122786, WO 2008/020079, WO 2008/142164 or WO 2009/068627.

The invention also relates to a pharmaceutical composition that comprises at least one compound or polypeptide of the invention, and optionally at least one pharmaceutically acceptable carrier, diluent or excipient. Such preparations, carriers, excipients and diluents may generally be as described in the published patent applications of Ablynx N.V., such as for example WO 04/041862, WO 2006/122786, WO 2008/020079, WO 2008/142164 or WO 2009/068627.

However, since the compounds or polypeptides of the invention have an increased half-life, they are preferably administered to the circulation. As such, they can be administered in any suitable manner that allows the compound or polypeptide of the invention to enter the circulation, such as intravenously, via injection or infusion, or in any other suitable manner (including oral administration, subcutaneous administration, intramuscular administration, administration through the skin, intranasal administration, administration via the lungs, etc.). Suitable methods and routes of administration will be clear to the skilled person, again for example also from the teaching of the published patent applications of Ablynx N.V., such as for example WO 04/041862, WO 2006/122786, WO 2008/020079, WO 2008/142164 or WO 2009/068627.

Thus, in another aspect, the invention relates to a method for the prevention and/or treatment of at least one disease or disorder that can be prevented or treated by the use of a compound or polypeptide of the invention, which method comprises administering, to a subject in need thereof, a pharmaceutically active amount of a compound or polypeptide of the invention, and/or of a pharmaceutical composition comprising the same. The diseases and disorders that can be prevented or treated by the use of a compound or polypeptide of the invention as described herein will generally be the same as the diseases and disorders that can be prevented or treated by the use of the therapeutic moiety or moieties that is/are present in the compound or polypeptide of the invention.

In the context of the present invention, the term “prevention and/or treatment” not only comprises preventing and/or treating the disease, but also generally comprises preventing the onset of the disease, slowing or reversing the progress of disease, preventing or slowing the onset of one or more symptoms associated with the disease, reducing and/or alleviating one or more symptoms associated with the disease, reducing the severity and/or the duration of the disease and/or of any symptoms associated therewith and/or preventing a further increase in the severity of the disease and/or of any symptoms associated therewith, preventing, reducing or reversing any physiological damage caused by the disease, and generally any pharmacological action that is beneficial to the patient being treated.

The subject to be treated may be any warm-blooded animal, but is in particular a mammal, and more in particular a human being. As will be clear to the skilled person, the subject to be treated will in particular be a person suffering from, or at risk from, the diseases and disorders mentioned herein.

In another embodiment, the invention relates to a method for immunotherapy, and in particular for passive immunotherapy, which method comprises administering, to a subject suffering from or at risk of the diseases and disorders mentioned herein, a pharmaceutically active amount of a compound or polypeptide of the invention, and/or of a pharmaceutical composition comprising the same.

The compound or polypeptide of the invention and/or the compositions comprising the same are administered according to a regime of treatment that is suitable for preventing and/or treating the disease or disorder to be prevented or treated. The clinician will generally be able to determine a suitable treatment regimen, depending on factors such as the disease or disorder to be prevented or treated, the severity of the disease to be treated and/or the severity of the symptoms thereof, the specific polypeptide of the invention to be used, the specific route of administration and pharmaceutical formulation or composition to be used, the age, gender, weight, diet, general condition of the patient, and similar factors well known to the clinician.

Generally, the treatment regimen will comprise the administration of one or more compounds or polypeptides of the invention, or of one or more compositions comprising the same, in one or more pharmaceutically effective amounts or doses. The specific amount(s) or doses to be administered can be determined by the clinician, again based on the factors cited above.

Generally, for the prevention and/or treatment of the diseases and disorders mentioned herein and depending on the specific disease or disorder to be treated, the potency and/or the half-life of the compounds or polypeptides of the invention to be used, the specific route of administration and the specific pharmaceutical formulation or composition used, the compounds or polypeptides of the invention will generally be administered in an amount between 1 gram and 0.01 microgram per kg body weight per day, preferably between 0.1 gram and 0.1 microgram per kg body weight per day, such as about 1, 10, 100 or 1000 microgram per kg body weight per day, either continuously (e.g. , by infusion), as a single daily dose or as multiple divided doses during the day. The clinician will generally be able to determine a suitable daily dose, depending on the factors mentioned herein. It will also be clear that in specific cases, the clinician may choose to deviate from these amounts, for example on the basis of the factors cited above and his expert judgment. Generally, some guidance on the amounts to be administered can be obtained from the amounts usually administered for comparable conventional antibodies or antibody fragments against the same target administered via essentially the same route, taking into account however differences in affinity/avidity, efficacy, biodistribution, half-life and similar factors well known to the skilled person.

Also, as the compounds of the invention contain a half-life extending serum albumin binder of the invention, they do not need to be administered essentially continuously (e.g. by infusion), but they can be administered at suitable intervals (to be determined by the skilled person). For example, they can be administered (at a suitable dose) once every two days, once every four days, once weekly, once every two weeks and in some cases once every four weeks or even less frequently, for example by injection or infusion.

One aspect of the invention relates to a pharmaceutical composition comprising at least one compound or polypeptide of the invention wherein said composition is intended for administration at an interval between once weekly and once every 4 weeks, and in particular between once every 7 days and once every 21 days, such as once every 7 days or 14 days.

Usually, in the above method, a single polypeptide of the invention will be used. It is however within the scope of the invention to use two or more polypeptides of the invention in combination.

The polypeptides of the invention may also be used in combination with one or more further pharmaceutically active compounds or principles, i.e., as a combined treatment regimen, which may or may not lead to a synergistic effect. Again, the clinician will be able to select such further compounds or principles, as well as a suitable combined treatment regimen, based on the factors cited above and his expert judgement.

In particular, the polypeptides of the invention may be used in combination with other pharmaceutically active compounds or principles that are or can be used for the prevention and/or treatment of the diseases and disorders that can be prevented or treated with the fusion proteins or constructs of the invention, and as a result of which a synergistic effect may or may not be obtained.

The effectiveness of the treatment regimen used according to the invention may be determined and/or followed in any manner known per se for the disease or disorder involved, as will be clear to the clinician. The clinician will also be able, where appropriate and or a case-by-case basis, to change or modify a particular treatment regimen, so as to achieve the desired therapeutic effect, to avoid, limit or reduce unwanted side-effects, and/or to achieve an appropriate balance between achieving the desired therapeutic effect on the one hand and avoiding, limiting or reducing undesired side effects on the other hand.

Generally, the treatment regimen will be followed until the desired therapeutic effect is achieved and/or for as long as the desired therapeutic effect is to be maintained. Again, this can be determined by the clinician.

Other aspects, embodiments, advantages and applications of the invention will become clear from the further description herein.

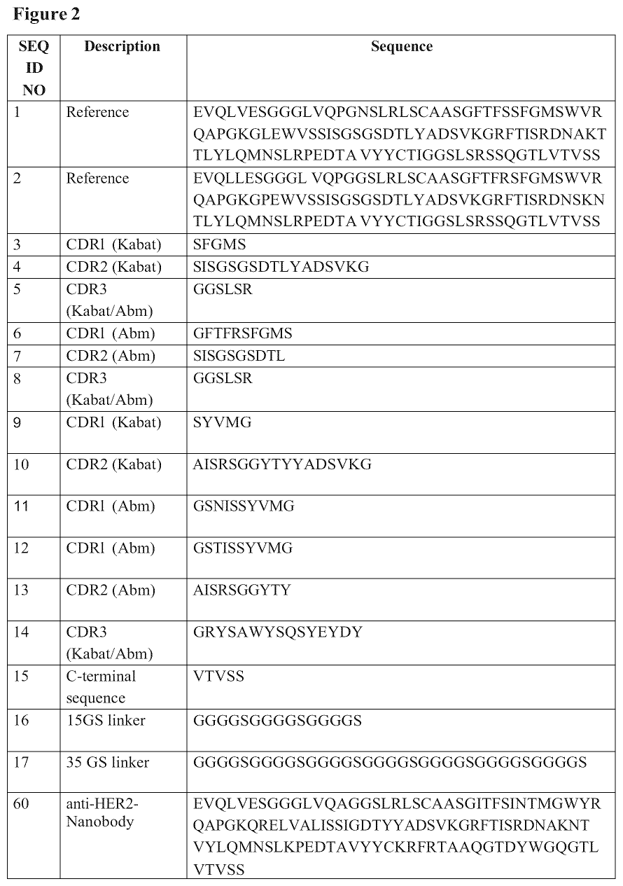

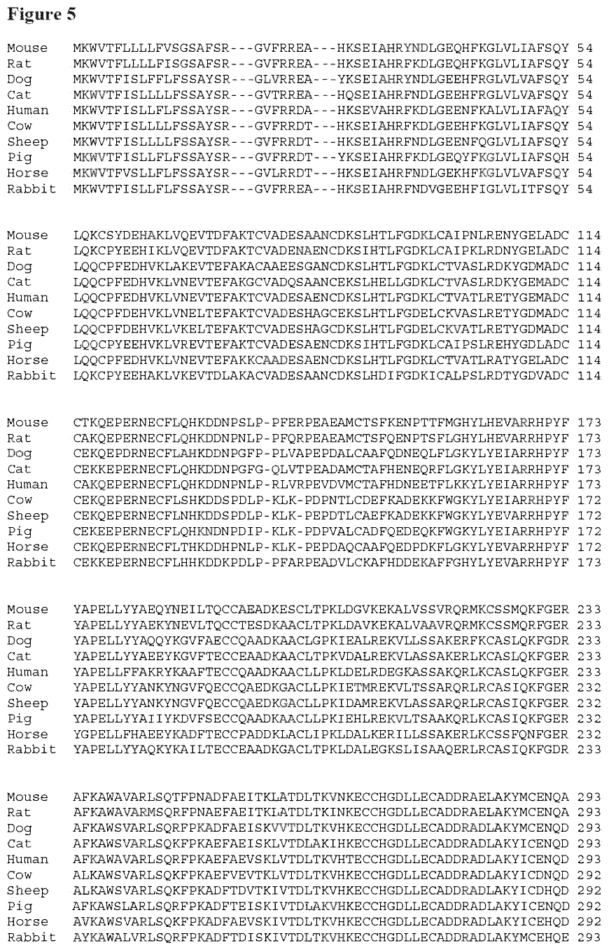

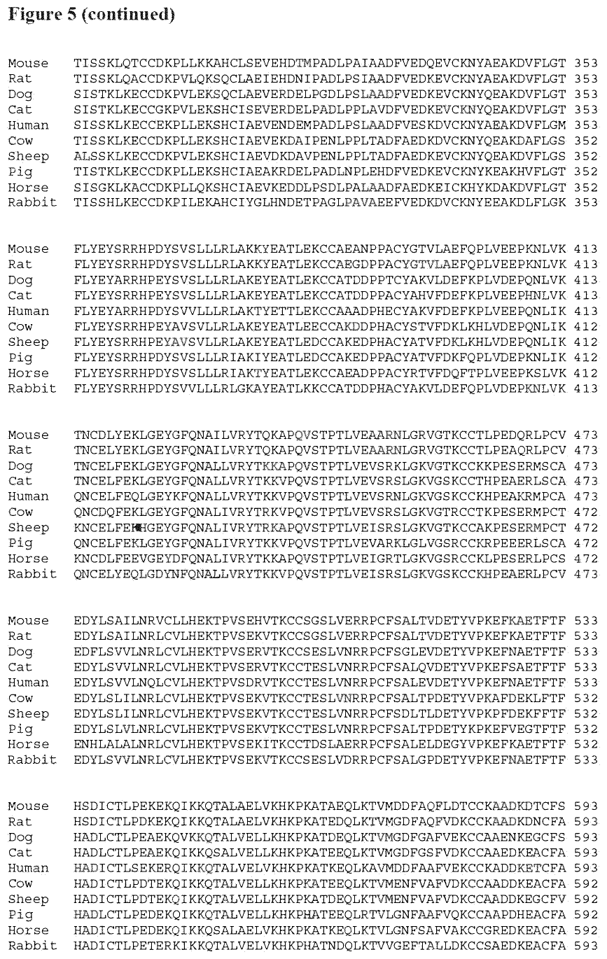

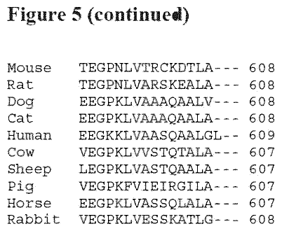

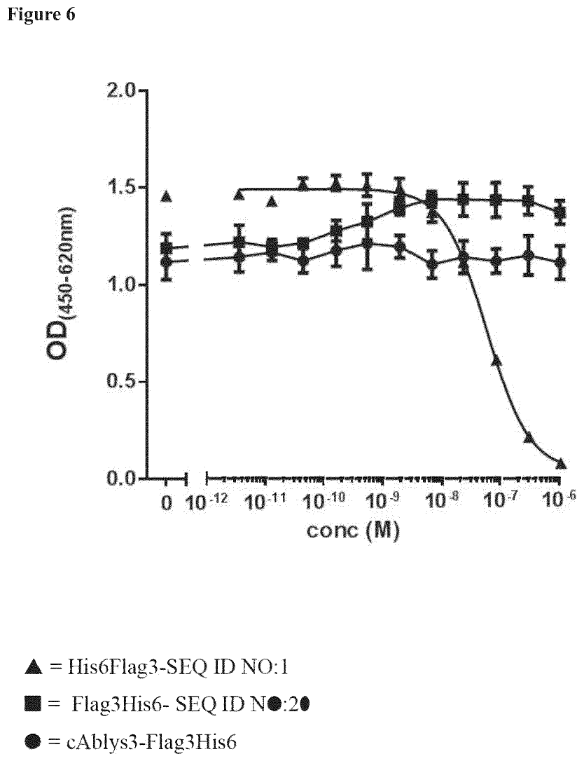

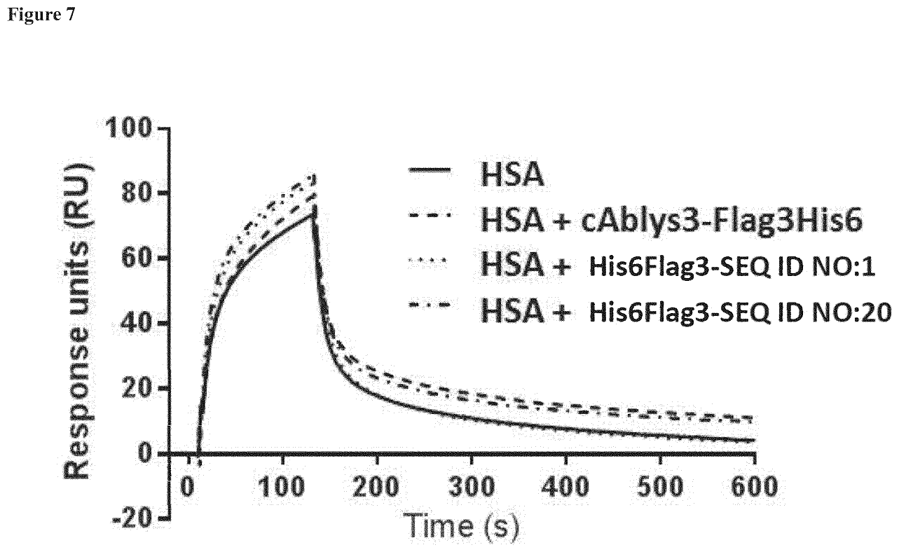

The invention will now be further described by means of the following non-limiting preferred aspects, examples and figures, in which:
FIG. 1 is a table listing some of the amino acid positions that will be specifically referred to herein and their numbering according to some alternative numbering systems (such as Aho and IMGT);
FIG. 2 lists amino acid sequences referred to herein;
FIGS. 3A and 3B show an alignment of the sequences of SEQ ID NOs: 18 to 20 (invention) with the prior art sequences of SEQ ID NOs: 1 and 2 (these sequence identifiers are also provided on the left side of the presented alignment);
FIG. 4 shows an alignment of SEQ ID NOs: 18 to 59 (these sequence identifiers are also provided on the left side of the presented alignment);
FIG. 5 gives an alignment of serum albumin from different species of mammal. Mouse: SEQ ID NO: 70; Rat: SEQ ID NO: 71; Dog: SEQ ID NO: 74; Cat: SEQ ID NO: 75; Human: SEQ ID NO: 69; Cow: SEQ ID NO: 72; Sheep: SEQ ID NO: 76; Pig: SEQ ID NO: 73; Horse: SEQ ID NO: 77; Rabbit: SEQ ID NO: 78.
FIG. 6 is a graph showing binding of SEQ ID NO: 1 (reference)-cMycHis6 to coated HSA in presence of the serum albumin binder of SEQ ID NO:20 (invention). 1.5 nM SEQ ID NO:1-cMycHis6 and concentration series of His6Flag3-SEQ ID NO:1 (reference), Flag3His6-SEQ ID NO:20 (invention) or cAblys3-Flag3His6 (reference) were incubated on coated HSA. Bound SEQ ID NO:1-cMycHis6 was detected with goat anti-cMyc and HRP-labelled rabbit anti-goat antibodies.
FIG. 7 is a graph showing binding of HSA to FcRn in presence of the serum albumin binder of SEQ ID NO:20 (invention). Human FcRn-human β2 microglobulin heterodimer was immobilized on CM5 chip. Binding of 1 μM HSA in absence or presence of 2 μM Nanobody in 50 mM NaPO4+150 mM NaCl+0.05% Tween-20 pH 6.0 was monitored on a Biacore T100 instrument.

The entire contents of all of the references (including literature references, issued patents, published patent applications, and co pending patent applications) cited throughout this application are hereby expressly incorporated by reference, in particular for the teaching that is referenced hereinabove.

### EXPERIMENTAL PART

### Example 1

### Affinity for Serum Albumin

The affinity of the serum albumin binder of SEQ ID NO:19 (invention) for human (Sigma-Aldrich A3782), cynomolgus monkey (generated in-house), mouse (Albumin Bioscience 2601), rat (Sigma-Aldrich A4538), rabbit (Sigma-Aldrich A0764), guinea pig (Gentaur GPSA62), pig (Sigma-Aldrich A4414), sheep (Sigma-Aldrich A3264), dog (Abcam 119814) and bovine (Sigma-Aldrich A3059) serum albumin (SA) was measured via Surface Plasmon Resonance (SPR) on a ProteOn XPR36 (BioRad) instrument. Serum albumin was immobilized via amine coupling on GLC ProteOn chip using ProteOn Amine Coupling Kit (BioRad). Different concentrations (300 nM, 100 nM, 33.3 nM, 11.1 nM, 3.7 nM and 1.23 nM) of The serum albumin binder of SEQ ID NO:19 (invention) was injected in HBS-P+ pH 7.4 buffer (GE Healthcare) at 45 μL/min for 120 s, followed by dissociation for 900 s. There was no or very low binding observed for SEQ ID NO: 19 on sheep and bovine SA. The affinity of SEQ ID NO: 19 for human, cynomolgus monkey, rat, mouse and guinea pig SA was in the pM range (Table 1). In addition, SEQ ID NO:19 is cross-reactive with dog SA and also binds with weak affinity to pig and rabbit SA.

TABLE 1
Kinetic parameters for binding of The serum albumin binder
of SEQ ID NO: 19 (invention) on SA from different species.

| | SEQ ID NO: 19 (invention) | | | SEQ ID NO: 1 (reference) | | |
|---|---|---|---|---|---|---|
| | ka | | | ka | | |
| | (s-1 | Kd | KD | (s-1 | Kd | KD |
| SA | M-1) | (s-1) | (M) | M-1) | (s-1) | (M) |
| human | 7.9E+05 | 3.7E−05 | 4.6E−11 | 4.9E+05 | 1.6E−03 | 3.3E−09 |
| cyno | 8.3E+05 | 1.4E−05 | 1.6E−11 | 4.6E+05 | 1.4E−03 | 3.1E−09 |
| rat | 1.2E+06 | 1.5E−05 | 1.2E−11 | 3.9E+05 | 2.6E−01 | 6.7E−07 |
| mouse | 9.4E+05 | 1.5E−05 | 1.6E−11 | 6.6E+05 | 3.0E−02 | 3.9E−08 |
| guinea pig | 9.4E+05 | 5.6E−05 | 5.9E−11 | 9.4E+05 | 1.9E−02 | 2.0E−08 |

| | | | | |
|---|---|---|---|---|
| dog | 9.0E+05 | 2.5E−03 | 2.8E−09 | no binding |
| pig | 1.2E+05 | 3.6E−02 | 2.9E−07 | no binding |

The serum albumin binder of SEQ ID NO: 19 (invention) was injected at different concentrations on immobilized SA on a ProteOn instrument.
Binding and dissociation kinetics were analysed at pH 7.4.
Kinetic parameters for SEQ ID NO: 1 are listed as a reference and were determined in separate experiments.

The long half-life of albumin in blood is mainly driven by two characteristics: (i) the large size (65 kDa) of albumin limits its glomerular filtration and (ii) albumin binds to FcRn at low pH (pH 6), which protects albumin from degradation in the lysosomes after passive endocytosis in endothelial and epithelial cells, by recycling from early endosome back to the extracellular environment. For albumin-binding Nanobodies to result in long serum half-life through albumin binding and subsequent recycling, these should stay bound to albumin in the pH range from 5.0 to 7.4. The dissociation rate of the serum albumin binder of SEQ ID NO: 19 (invention) from HSA at pH 5, pH 6 and pH 7.4 was measured on a ProteOn instrument as described above, including SEQ ID NO: 1 as a reference. The serum albumin binders of SEQ ID NO:19 (invention) and SEQ ID NO:1 were injected at 500 nM and 300 nM respectively in HBS-P+ pH 7.4 buffer. Dissociation buffers were 50 mM NaOAc/HOAc+150 mM NaCl+0.05% Tween-20 pH 5.0, 50 mM NaOAc/HOAc+150 mM NaCl+0.05% Tween-20 pH 6.0 and HBS-P+pH 7.4 respectively. Dissociation was analysed for 2700 s. Dissociation rates for SEQ ID NO:19 do not differ significantly across the pH range from 5.0 to 7.4 (Table 2).

TABLE 2
Dissociation rate of the serum albumin binder of SEQ
ID NO: 19 (invention) from HSA at different pH.

| | kd (s−1) | |
|---|---|---|
| pH | SEQ ID NO: 19 (invention) | SEQ ID NO: 1 (reference) |
| pH 7.4 | 6.0E−05 | 1.3E−03 |
| pH 6.0 | 6.7E−05 | 9.2E−04 |
| pH 5.0 | 7.5E−05 | 1.1E−03 |

SEQ ID NO: 19 (invention) and SEQ ID NO: 1 (reference) were injected on immobilized HSA. Dissociation was monitored at pH 5.0, 6.0 and 7.4 on a ProteOn instrument.

### Example 2

### Epitope

Epitope binning was analysed in a competition ELISA. Human serum albumin was coated at 125 ng/ml in PBS at 4° C. overnight. After blocking with PBS +1% casein, 1.5 nM SEQ ID NO:1-cMycHis6 and a concentration series of competitors (His6Flag3-SEQ ID NO:20, His6Flag3-SEQ ID NO:1 as positive control or hen egg lysozyme binding single domain antibody cAblys3-Flag3His6 as negative control) were added. Bound SEQ ID NO:1-cMycHis6 was detected with goat anti-cMyc (Abcam ab19234) and HRP-labelled rabbit anti-goat (Genway 18-511-244226) antibodies. The albumin binder of SEQ ID NO:20 and SEQ ID NO:1 do not bind identical epitopes on HSA (FIG. 6).

### Example 3

Interference with Interaction Between SA and FcRn

For the serum albumin binder of SEQ ID NO:19 (invention) to provide long half-life via albumin binding and subsequent recycling, it should not interfere with the binding of albumin to FcRn. This was analysed in SPR on a Biacore T100 (GE Healthcare) instrument. Human FcRn-human β2 microglobulin heterodimer (Sino Biological CT009-H08H) was immobilized on CM5 chip via standard amine coupling (Biacore amine coupling kit). A mixture of 1 μM HSA and 2 μM Nanobody (His6Flag3-SEQ ID NO:20, His6Flag3-SEQ ID NO: 1 or cAblys3-Flag3His6) in 50 mM NaPO4 +150 mM NaCl +0.05% Tween-20 pH 6.0 was injected at 10 μl/min for 120 s, followed by dissociation for 600 s. Binding curves were qualitatively compared with binding curve of 1 μM HSA in absence of Nanobody. The albumin binder of SEQ ID NO:20 (invention) did not interfere with the binding of HSA to FcRn (FIG. 7).

### Example 4

### PK Profile in Dog

Pharmacokinetics of a trivalent Nanobody construct (SEQ ID NO:68) after single i.v. dose are studied in dog. The trivalent construct was produced in Pichia pastoris and comprises two Nanobodies against respiratory syncytial virus (RSV) with the albumin binding Nanobody of SEQ ID NO: 19 at the C-terminal end (in which the three Nanobodies are linked to each other using (Gly4Ser)7 linkers).

The pharmacokinetic profile of the construct is evaluated in a pharmacokinetic study (non-crossover, single dose) in twelve fasted healthy male Beagle dogs (healthy as determined by a pre-study health examination). The construct is administered intravenously.

The animals are fasted (with uninterrupted access to water) overnight prior to dosing, and prior to collection of blood samples at pre-dose, 24, 168, and 336 hours after dosing (total fasting time no longer than 24 hours). Food is returned 4 hours post-dose, or immediately after blood collection at 24, 168, and 336 hours. Body weight is also recorded and tracked during the study.

1 ml of whole blood (or 2-3 ml pre-dose and at 24, 168 and 336 hours) for plasma is collected from a peripheral vessel pre-dose and at 5, 15, 30 min and 1, 4, 8, 12, 24, 72, 168, 240, 336, 504, and 672 hours post initial dose. Collected samples for serum processing are kept at room temperature and are allowed to clot. Blood is be centrifuged at 10,000 rpm for 2 minutes using a refrigerated centrifuge (set to maintain 4°° C.). Samples are frozen within 30 minutes from collection until they are submitted for analysis (including measurement of concentration of the relevant Nanobody construct).

1. A method for preventing or treating a disease or condition in a subject, comprising administering to the subject an immunoglobulin single variable domain (ISVD), or a polypeptide comprising said ISVD, wherein the ISVD is capable of binding to serum albumin and comprises:
a CDR1 (according to Abm) that is the amino acid sequence GSNISSYVMG (SEQ ID NO:11) or GSTISSYVMG (SEQ ID NO:12) or an amino acid sequence that has 2 or 1 amino acid difference(s) with the amino acid sequence of SEQ ID NO: 11 or SEQ ID NO: 12; and
a CDR2 (according to Abm) that is the amino acid sequence AISRSGGYTY (SEQ ID NO: 13) or an amino acid sequence that has 3, 2 or 1 amino acid difference(s) with the amino acid sequence of SEQ ID NO: 13; and
a CDR3 (according to Abm) that is the amino acid sequence GRYSAWYSQSYEYDY (SEQ ID NO: 14) or an amino acid sequence that has 3, 2 or 1 amino acid difference(s) with the amino acid sequence of SEQ ID NO: 14,
optionally wherein the serum albumin is human serum albumin.

2. The method according to claim 1, wherein the ISVD comprises:
a CDR1 (according to Abm) that is the amino acid sequence GSNISSYVMG (SEQ ID NO:11) or GSTISSYVMG (SEQ ID NO:12); and
a CDR2 (according to Abm) that is the amino acid sequence AISRSGGYTY (SEQ ID NO: 13); and
a CDR3 (according to Abm) that is the amino acid sequence GRYSAWYSQSYEYDY (SEQ ID NO: 14).

3. The method according to claim 1, wherein the ISVD can bind to human serum albumin with an affinity better than 100 nM, optionally better than 50nM, as determined using surface plasmon resonance.

4. The method according to claim 1, wherein the ISVD is a heavy-chain immunoglobulin single variable domain.

5. The method according to claim 1, wherein the ISVD is a VHH, a humanized VHH or a camelized VH, optionally wherein the camelized VH is a camelized human VH.

6. The method according to claim 1, wherein the ISVD, or the polypeptide comprising the ISVD
has a serum half-life in human (expressed as t1/2 beta) that is more than 6 hours, 12 hours, 24 hours, or 72 hours;
and/or.

7. The method according to claim 1, wherein the ISVD has:
a degree of sequence identity with the sequence of SEQ ID NO: 18 and/or 19 of at least 85%, at least 90%, or at least 95%;
and/or:
-no more than 7, no more than 5, no more than 3, no more than 2, or no more than 1 amino acid differences with the sequence of SEQ ID NO: 18 and/or 19;
wherein the CDRs and any C-terminal extension that may be present are not taken into account for determining the degree of sequence identity or the amino acid differences.

8. The method according to claim 1, wherein the ISVD has:
a degree of sequence identity with the sequence of SEQ ID NO: 18 of at least 85%, at least 90%, or at least 95%;
and/or:
no more than 7, no more than 5, no more than 3, no more than 2, or no more than 1 amino acid differences with the sequence of SEQ ID NO: 18;
wherein the CDRs and any C-terminal extension that may be present are not taken into account for determining the degree of sequence identity or the amino acid differences.

9. The method according to claim 1, wherein the ISVD has:
a degree of sequence identity with the sequence of SEQ ID NO: 19 of at least 85%, at least 90%, or at least 95%;
and/or:
no more than 7, no more than 5, no more than 3, no more than 2, or no more than 1 amino acid differences with the sequence of SEQ ID NO: 19;
wherein the CDRs and any C-terminal extension that may be present are not taken into account for determining the degree of sequence identity or the amino acid differences.

10. The method according to claim 1, wherein the ISVD contains, compared to the sequence of SEQ ID NO: 18, one or more mutations that reduce the binding by pre-existing antibodies.

11. The method according to claim 1, wherein the ISVD is a VHH and that contains, compared to the sequence of SEQ ID NO:18, one or more humanizing substitutions.

12. The method according to claim 1, wherein the ISVD comprises the amino acid sequence of any one of SEQ ID NOs: 18-59.

13. The method according to claim 1, wherein a polypeptides the ISVD.

14. The method according to claim 13, wherein the polypeptide comprises at least one therapeutic moiety or entity.

15. The method according to claim 13, wherein the polypeptide is a fusion protein.

16. The method according to claim 13, wherein the polypeptide has a serum half-life in human (expressed as t1/2 beta) that is more than 6 hours, more than 12 hours, more than 24 hours, or more than 72 hours.

17. The method according to claim 13 wherein the polypeptide is such that:
when the polypeptide comprises a C-terminal ISVD, it further comprises a C-terminal extension (X)ₙ; and/or
when the polypeptide comprises a C-terminal ISVD, said C-terminal ISVD contains one or more mutations that reduce the binding of pre-existing antibodies; and/or
when the polypeptide comprises a N-terminal ISVD, said N-terminal ISVD contains a D at position 1; and/or
when the polypeptide comprises a N-terminal ISVD, said N-terminal ISVD contains a D or an EID at position 1; and/or
essentially all of the ISVDs present in said polypeptide, contain one or more mutations that reduce the binding of pre-existing antibodies.

18. The method according to claim 13, comprising administering to the subject a pharmaceutical composition comprising the ISVD or the polypeptide comprising said ISVD.

19. A nucleic acid that encodes an immunoglobulin single variable domain (ISVD), or a polypeptide comprising said ISVD, wherein the ISVD is capable of binding to serum albumin and comprises:
a CDR1 (according to Abm) that is the amino acid sequence GSNISSYVMG (SEQ ID NO:11) or GSTISSYVMG (SEQ ID NO:12) or an amino acid sequence that has 2 or 1 amino acid difference(s) with the amino acid sequence of SEQ ID NO: 11 or SEQ ID NO: 12; and
a CDR2 (according to Abm) that is the amino acid sequence AISRSGGYTY (SEQ ID NO: 13) or an amino acid sequence that has 3, 2 or 1 amino acid difference(s) with the amino acid sequence of SEQ ID NO: 13; and
a CDR3 (according to Abm) that is the amino acid sequence GRYSAWYSQSYEYDY (SEQ ID NO: 14) or an amino acid sequence that has 3, 2 or 1 amino acid difference(s) with the amino acid sequence of SEQ ID NO: 14,
optionally wherein the serum albumin is human serum albumin,
optionally wherein the nucleic acid is a genetic construct.

20. A host or host cell that comprises the nucleic acid according to claim 19.

21. A method for preparing a polypeptide comprising cultivating or maintaining a host cell according to claim 20 under conditions such that said host cell produces or expresses the ISVD or polypeptide, and optionally further comprising isolating the ISVD or polypeptide.


SEQUENCE LISTING (78 sequences)

SEQ ID NO: 1 - protein, 115 aa, synthetic construct [sequence omitted]

SEQ ID NO: 2 - protein, 115 aa, synthetic construct [sequence omitted]

SEQ ID NO: 3 - protein, 5 aa, synthetic construct
  REGION 1..5: note = CDR1 (Kabat)
SFGMS

SEQ ID NO: 4 - protein, 17 aa, synthetic construct
  REGION 1..17: note = CDR2 (Kabat)
SISGSGSDTLYADSVKG

SEQ ID NO: 5 - protein, 6 aa, synthetic construct
  REGION 1..6: note = CDR3 (Kabat/Abm)
GGSLSR

SEQ ID NO: 6 - protein, 10 aa, synthetic construct
  REGION 1..10: note = CDR1 (Abm)
GFTFRSFGMS

SEQ ID NO: 7 - protein, 10 aa, synthetic construct
  REGION 1..10: note = CDR2 (Abm)
SISGSGSDTL

SEQ ID NO: 8 - protein, 6 aa, synthetic construct
  REGION 1..6: note = CDR3 (Kabat/Abm)
GGSLSR

SEQ ID NO: 9 - protein, 5 aa, synthetic construct
  REGION 1..5: note = CDR1 (Kabat)
SYVMG

SEQ ID NO: 10 - protein, 17 aa, synthetic construct
  REGION 1..17: note = CDR2 (Kabat)
AISRSGGYTYYADSVKG

SEQ ID NO: 11 - protein, 10 aa, synthetic construct
  REGION 1..10: note = CDR1 (Abm)
GSNISSYVMG

SEQ ID NO: 12 - protein, 10 aa, synthetic construct
  REGION 1..10: note = CDR1 (Abm)
GSTISSYVMG

SEQ ID NO: 13 - protein, 10 aa, synthetic construct
  REGION 1..10: note = CDR2 (Abm)
AISRSGGYTY

SEQ ID NO: 14 - protein, 15 aa, synthetic construct
  REGION 1..15: note = CDR3 (Abm)
GRYSAWYSQSYEYDY

SEQ ID NO: 15 - protein, 5 aa, synthetic construct
  REGION 1..5: note = C-terminal sequence
VTVSS

SEQ ID NO: 16 - protein, 15 aa, synthetic construct
  REGION 1..15: note = 15 GS linker
GGGGSGGGGSGGGGS

SEQ ID NO: 17 - protein, 35 aa, synthetic construct
  REGION 1..35: note = 35 GS linker
GGGGSGGGGSGGGGSGGGGSGGGGSGGGGSGGGGS

SEQ ID NO: 18 - protein, 124 aa, synthetic construct
  REGION 1..124: note = ISVD
EVQLVESGGGLVQAGGSLRLSCAASGSNISSYVMGWFRRAPGKEREFVAAISRSGGYTYYADSVKGRFTISRDNAKKTAYLQMNSLEPEDTAVYYCAAGRYSAWYSQSYEYDYWGQGTLVTVSS

SEQ ID NO: 19 - protein, 124 aa, synthetic construct
  REGION 1..124: note = ISVD
EVQLVESGGGVVQPGGSLRLSCAASGSTISSYVMGWFRRAPGKEREFVAAISRSGGYTYYADSVKGRFTISRDNSKKTAYLQMNSLRPEDTALYYCAAGRYSAWYSQSYEYDYWGQGTLVTVSS

SEQ ID NO: 20 - protein, 124 aa, synthetic construct
  REGION 1..124: note = ISVD
EVQLVESGGGVVQPGGSLRLSCAASGSNISSYVMGWFRRAPGKEREFVAAISRSGGYTYYADSVKGRFTISRDNSKKTAYLQMNSLRPEDTALYYCAAGRYSAWYSQSYEYDYWGQGTLVTVSS

SEQ ID NO: 21 - protein, 124 aa, synthetic construct
  REGION 1..124: note = ISVD
EVQLVESGGGVVQPGGSLRLSCAASGSTISSYVMGWFRQAPGKEREFVAAISRSGGYTYYADSVKGRFTISRDNSKKTAYLQMNSLRPEDTALYYCAAGRYSAWYSQSYEYDYWGQGTLVTVSS

SEQ ID NO: 22 - protein, 124 aa, synthetic construct
  REGION 1..124: note = ISVD
EVQLVESGGGVVQPGGSLRLSCAASGSTISSYVMGWFRRAPGKEREFVAAISRSGGYTYYADSVKGRFTISRDNSKNTAYLQMNSLRPEDTALYYCAAGRYSAWYSQSYEYDYWGQGTLVTVSS

SEQ ID NO: 23 - protein, 124 aa, synthetic construct
  REGION 1..124: note = ISVD
EVQLVESGGGVVQPGGSLRLSCAASGSTISSYVMGWFRRAPGKEREFVAAISRSGGYTYYADSVKGRFTISRDNSKKTVYLQMNSLRPEDTALYYCAAGRYSAWYSQSYEYDYWGQGTLVTVSS

SEQ ID NO: 24 - protein, 124 aa, synthetic construct
  REGION 1..124: note = ISVD
EVQLVESGGGVVQPGGSLRLSCAASGSTISSYVMGWFRQAPGKEREFVAAISRSGGYTYYADSVKGRFTISRDNSKNTAYLQMNSLRPEDTALYYCAAGRYSAWYSQSYEYDYWGQGTLVTVSS

SEQ ID NO: 25 - protein, 124 aa, synthetic construct
  REGION 1..124: note = ISVD
EVQLVESGGGVVQPGGSLRLSCAASGSTISSYVMGWFRQAPGKEREFVAAISRSGGYTYYADSVKGRFTISRDNSKKTVYLQMNSLRPEDTALYYCAAGRYSAWYSQSYEYDYWGQGTLVTVSS

SEQ ID NO: 26 - protein, 124 aa, synthetic construct
  REGION 1..124: note = ISVD
EVQLVESGGGVVQPGGSLRLSCAASGSTISSYVMGWFRRAPGKEREFVAAISRSGGYTYYADSVKGRFTISRDNSKNTVYLQMNSLRPEDTALYYCAAGRYSAWYSQSYEYDYWGQGTLVTVSS

SEQ ID NO: 27 - protein, 124 aa, synthetic construct
  REGION 1..124: note = ISVD
EVQLVESGGGVVQPGGSLRLSCAASGSTISSYVMGWFRQAPGKEREFVAAISRSGGYTYYADSVKGRFTISRDNSKNTVYLQMNSLRPEDTALYYCAAGRYSAWYSQSYEYDYWGQGTLVTVSS

SEQ ID NO: 28 - protein, 124 aa, synthetic construct; identical to SEQ ID NO: 23
  REGION 1..124: note = ISVD

SEQ ID NO: 29 - protein, 124 aa, synthetic construct; identical to SEQ ID NO: 25
  REGION 1..124: note = ISVD

SEQ ID NO: 30 - protein, 124 aa, synthetic construct; identical to SEQ ID NO: 26
  REGION 1..124: note = ISVD

SEQ ID NO: 31 - protein, 124 aa, synthetic construct; identical to SEQ ID NO: 27
  REGION 1..124: note = ISVD

SEQ ID NO: 32 - protein, 125 aa, synthetic construct
  REGION 1..125: note = ISVD
EVQLVESGGGLVQAGGSLRLSCAASGSNISSYVMGWFRRAPGKEREFVAAISRSGGYTYYADSVKGRFTISRDNAKKTAYLQMNSLEPEDTAVYYCAAGRYSAWYSQSYEYDYWGQGTLVTVSSA

SEQ ID NO: 33 - protein, 125 aa, synthetic construct
  REGION 1..125: note = ISVD
EVQLVESGGGVVQPGGSLRLSCAASGSTISSYVMGWFRRAPGKEREFVAAISRSGGYTYYADSVKGRFTISRDNSKKTAYLQMNSLRPEDTALYYCAAGRYSAWYSQSYEYDYWGQGTLVTVSSA

SEQ ID NO: 34 - protein, 125 aa, synthetic construct
  REGION 1..125: note = ISVD
EVQLVESGGGVVQPGGSLRLSCAASGSNISSYVMGWFRRAPGKEREFVAAISRSGGYTYYADSVKGRFTISRDNSKKTAYLQMNSLRPEDTALYYCAAGRYSAWYSQSYEYDYWGQGTLVTVSSA

SEQ ID NO: 35 - protein, 125 aa, synthetic construct
  REGION 1..125: note = ISVD
EVQLVESGGGVVQPGGSLRLSCAASGSTISSYVMGWFRQAPGKEREFVAAISRSGGYTYYADSVKGRFTISRDNSKKTAYLQMNSLRPEDTALYYCAAGRYSAWYSQSYEYDYWGQGTLVTVSSA

SEQ ID NO: 36 - protein, 125 aa, synthetic construct
  REGION 1..125: note = ISVD
EVQLVESGGGVVQPGGSLRLSCAASGSTISSYVMGWFRRAPGKEREFVAAISRSGGYTYYADSVKGRFTISRDNSKNTAYLQMNSLRPEDTALYYCAAGRYSAWYSQSYEYDYWGQGTLVTVSSA

SEQ ID NO: 37 - protein, 125 aa, synthetic construct
  REGION 1..125: note = ISVD
EVQLVESGGGVVQPGGSLRLSCAASGSTISSYVMGWFRRAPGKEREFVAAISRSGGYTYYADSVKGRFTISRDNSKKTVYLQMNSLRPEDTALYYCAAGRYSAWYSQSYEYDYWGQGTLVTVSSA

SEQ ID NO: 38 - protein, 125 aa, synthetic construct
  REGION 1..125: note = ISVD
EVQLVESGGGVVQPGGSLRLSCAASGSTISSYVMGWFRQAPGKEREFVAAISRSGGYTYYADSVKGRFTISRDNSKNTAYLQMNSLRPEDTALYYCAAGRYSAWYSQSYEYDYWGQGTLVTVSSA

SEQ ID NO: 39 - protein, 125 aa, synthetic construct
  REGION 1..125: note = ISVD
EVQLVESGGGVVQPGGSLRLSCAASGSTISSYVMGWFRQAPGKEREFVAAISRSGGYTYYADSVKGRFTISRDNSKKTVYLQMNSLRPEDTALYYCAAGRYSAWYSQSYEYDYWGQGTLVTVSSA

SEQ ID NO: 40 - protein, 125 aa, synthetic construct
  REGION 1..125: note = ISVD
EVQLVESGGGVVQPGGSLRLSCAASGSTISSYVMGWFRRAPGKEREFVAAISRSGGYTYYADSVKGRFTISRDNSKNTVYLQMNSLRPEDTALYYCAAGRYSAWYSQSYEYDYWGQGTLVTVSSA

SEQ ID NO: 41 - protein, 125 aa, synthetic construct
  REGION 1..125: note = ISVD
EVQLVESGGGVVQPGGSLRLSCAASGSTISSYVMGWFRQAPGKEREFVAAISRSGGYTYYADSVKGRFTISRDNSKNTVYLQMNSLRPEDTALYYCAAGRYSAWYSQSYEYDYWGQGTLVTVSSA

SEQ ID NO: 42 - protein, 125 aa, synthetic construct; identical to SEQ ID NO: 37
  REGION 1..125: note = ISVD

SEQ ID NO: 43 - protein, 125 aa, synthetic construct; identical to SEQ ID NO: 39
  REGION 1..125: note = ISVD

SEQ ID NO: 44 - protein, 125 aa, synthetic construct; identical to SEQ ID NO: 40
  REGION 1..125: note = ISVD

SEQ ID NO: 45 - protein, 125 aa, synthetic construct; identical to SEQ ID NO: 41
  REGION 1..125: note = ISVD

SEQ ID NO: 46 - protein, 124 aa, synthetic construct
  REGION 1..124: note = ISVD
DVQLVESGGGLVQAGGSLRLSCAASGSNISSYVMGWFRRAPGKEREFVAAISRSGGYTYYADSVKGRFTISRDNAKKTAYLQMNSLEPEDTAVYYCAAGRYSAWYSQSYEYDYWGQGTLVTVSS

SEQ ID NO: 47 - protein, 124 aa, synthetic construct
  REGION 1..124: note = ISVD
DVQLVESGGGVVQPGGSLRLSCAASGSTISSYVMGWFRRAPGKEREFVAAISRSGGYTYYADSVKGRFTISRDNSKKTAYLQMNSLRPEDTALYYCAAGRYSAWYSQSYEYDYWGQGTLVTVSS

SEQ ID NO: 48 - protein, 124 aa, synthetic construct
  REGION 1..124: note = ISVD
DVQLVESGGGVVQPGGSLRLSCAASGSNISSYVMGWFRRAPGKEREFVAAISRSGGYTYYADSVKGRFTISRDNSKKTAYLQMNSLRPEDTALYYCAAGRYSAWYSQSYEYDYWGQGTLVTVSS

SEQ ID NO: 49 - protein, 124 aa, synthetic construct
  REGION 1..124: note = ISVD
DVQLVESGGGVVQPGGSLRLSCAASGSTISSYVMGWFRQAPGKEREFVAAISRSGGYTYYADSVKGRFTISRDNSKKTAYLQMNSLRPEDTALYYCAAGRYSAWYSQSYEYDYWGQGTLVTVSS

SEQ ID NO: 50 - protein, 124 aa, synthetic construct
  REGION 1..124: note = ISVD
DVQLVESGGGVVQPGGSLRLSCAASGSTISSYVMGWFRRAPGKEREFVAAISRSGGYTYYADSVKGRFTISRDNSKNTAYLQMNSLRPEDTALYYCAAGRYSAWYSQSYEYDYWGQGTLVTVSS

SEQ ID NO: 51 - protein, 124 aa, synthetic construct
  REGION 1..124: note = ISVD
DVQLVESGGGVVQPGGSLRLSCAASGSTISSYVMGWFRRAPGKEREFVAAISRSGGYTYYADSVKGRFTISRDNSKKTVYLQMNSLRPEDTALYYCAAGRYSAWYSQSYEYDYWGQGTLVTVSS

SEQ ID NO: 52 - protein, 124 aa, synthetic construct
  REGION 1..124: note = ISVD
DVQLVESGGGVVQPGGSLRLSCAASGSTISSYVMGWFRQAPGKEREFVAAISRSGGYTYYADSVKGRFTISRDNSKNTAYLQMNSLRPEDTALYYCAAGRYSAWYSQSYEYDYWGQGTLVTVSS

SEQ ID NO: 53 - protein, 124 aa, synthetic construct
  REGION 1..124: note = ISVD
DVQLVESGGGVVQPGGSLRLSCAASGSTISSYVMGWFRQAPGKEREFVAAISRSGGYTYYADSVKGRFTISRDNSKKTVYLQMNSLRPEDTALYYCAAGRYSAWYSQSYEYDYWGQGTLVTVSS

SEQ ID NO: 54 - protein, 124 aa, synthetic construct
  REGION 1..124: note = ISVD
DVQLVESGGGVVQPGGSLRLSCAASGSTISSYVMGWFRRAPGKEREFVAAISRSGGYTYYADSVKGRFTISRDNSKNTVYLQMNSLRPEDTALYYCAAGRYSAWYSQSYEYDYWGQGTLVTVSS

SEQ ID NO: 55 - protein, 124 aa, synthetic construct
  REGION 1..124: note = ISVD
DVQLVESGGGVVQPGGSLRLSCAASGSTISSYVMGWFRQAPGKEREFVAAISRSGGYTYYADSVKGRFTISRDNSKNTVYLQMNSLRPEDTALYYCAAGRYSAWYSQSYEYDYWGQGTLVTVSS

SEQ ID NO: 56 - protein, 124 aa, synthetic construct; identical to SEQ ID NO: 51
  REGION 1..124: note = ISVD

SEQ ID NO: 57 - protein, 124 aa, synthetic construct; identical to SEQ ID NO: 53
  REGION 1..124: note = ISVD

SEQ ID NO: 58 - protein, 124 aa, synthetic construct; identical to SEQ ID NO: 54
  REGION 1..124: note = ISVD

SEQ ID NO: 59 - protein, 124 aa, synthetic construct; identical to SEQ ID NO: 55
  REGION 1..124: note = ISVD

SEQ ID NO: 60 - protein, 118 aa, synthetic construct
  REGION 1..118: note = anti-HER2-Nanobody
EVQLVESGGGLVQAGGSLRLSCAASGITFSINTMGWYRQAPGKQRELVALISSIGDTYYADSVKGRFTISRDNAKNTVYLQMNSLKPEDTAVYYCKRFRTAAQGTDYWGQGTLVTVSS

SEQ ID NO: 61 - protein, 278 aa, synthetic construct
  REGION 1..278: note = bivalent bispecific compound
DVQLVESGGGLVQAGGSLRLSCAASGITFSINTMGWYRQAPGKQRELVALISSIGDTYYADSVKGRFTISRDNAKNTVYLQMNSLKPEDTAVYYCKRFRTAAQGTDYWGQGTLVTVSSGGGGSGGGGSGGGGSGGGGSGGGGSGGGGSGGGGSEVQLVESGGGVVQPGGSLRLSCAASGSTISSYVMGWFRRAPGKEREFVAAISRSGGYTYYADSVKGRFTISRDNSKKTAYLQMNSLRPEDTALYYCAAGRYSAWYSQSYEYDYWGQGTLVTVSSA

SEQ ID NO: 62 - protein, 258 aa, synthetic construct
  REGION 1..258: note = bivalent bispecific compound
DVQLVESGGGLVQAGGSLRLSCAASGITFSINTMGWYRQAPGKQRELVALISSIGDTYYADSVKGRFTISRDNAKNTVYLQMNSLKPEDTAVYYCKRFRTAAQGTDYWGQGTLVTVSSGGGGSGGGGSGGGGSEVQLVESGGGVVQPGGSLRLSCAASGSTISSYVMGWFRRAPGKEREFVAAISRSGGYTYYADSVKGRFTISRDNSKKTAYLQMNSLRPEDTALYYCAAGRYSAWYSQSYEYDYWGQGTLVTVSSA

SEQ ID NO: 63 - protein, 258 aa, synthetic construct
  REGION 1..258: note = bivalent bispecific compound
DVQLVESGGGVVQPGGSLRLSCAASGSTISSYVMGWFRRAPGKEREFVAAISRSGGYTYYADSVKGRFTISRDNSKKTAYLQMNSLRPEDTALYYCAAGRYSAWYSQSYEYDYWGQGTLVTVSSGGGGSGGGGSGGGGSEVQLVESGGGLVQAGGSLRLSCAASGITFSINTMGWYRQAPGKQRELVALISSIGDTYYADSVKGRFTISRDNAKNTVYLQMNSLKPEDTAVYYCKRFRTAAQGTDYWGQGTLVTVSSA

SEQ ID NO: 64 - protein, 278 aa, synthetic construct
  REGION 1..278: note = bivalent bispecific compound
DVQLVESGGGVVQPGGSLRLSCAASGSTISSYVMGWFRRAPGKEREFVAAISRSGGYTYYADSVKGRFTISRDNSKKTAYLQMNSLRPEDTALYYCAAGRYSAWYSQSYEYDYWGQGTLVTVSSGGGGSGGGGSGGGGSGGGGSGGGGSGGGGSGGGGSEVQLVESGGGLVQAGGSLRLSCAASGITFSINTMGWYRQAPGKQRELVALISSIGDTYYADSVKGRFTISRDNAKNTVYLQMNSLKPEDTAVYYCKRFRTAAQGTDYWGQGTLVTVSSA

SEQ ID NO: 65 - protein, 431 aa, synthetic construct
  REGION 1..431: note = trivalent bispecific compound
DVQLVESGGGLVQAGGSLRLSCAASGITFSINTMGWYRQAPGKQRELVALISSIGDTYYADSVKGRFTISRDNAKNTVYLQMNSLKPEDTAVYYCKRFRTAAQGTDYWGQGTLVTVSSGGGGSGGGGSGGGGSGGGGSGGGGSGGGGSGGGGSEVQLVESGGGLVQAGGSLRLSCAASGITFSINTMGWYRQAPGKQRELVALISSIGDTYYADSVKGRFTISRDNAKNTVYLQMNSLKPEDTAVYYCKRFRTAAQGTDYWGQGTLVTVSSGGGGSGGGGSGGGGSGGGGSGGGGSGGGGSGGGGSEVQLVESGGGVVQPGGSLRLSCAASGSTISSYVMGWFRRAPGKEREFVAAISRSGGYTYYADSVKGRFTISRDNSKKTAYLQMNSLRPEDTALYYCAAGRYSAWYSQSYEYDYWGQGTLVTVSSA

SEQ ID NO: 66 - protein, 431 aa, synthetic construct
  REGION 1..431: note = trivalent bispecific compound
DVQLVESGGGLVQAGGSLRLSCAASGITFSINTMGWYRQAPGKQRELVALISSIGDTYYADSVKGRFTISRDNAKNTVYLQMNSLKPEDTAVYYCKRFRTAAQGTDYWGQGTLVTVSSGGGGSGGGGSGGGGSGGGGSGGGGSGGGGSGGGGSEVQLVESGGGVVQPGGSLRLSCAASGSTISSYVMGWFRRAPGKEREFVAAISRSGGYTYYADSVKGRFTISRDNSKKTAYLQMNSLRPEDTALYYCAAGRYSAWYSQSYEYDYWGQGTLVTVSSGGGGSGGGGSGGGGSGGGGSGGGGSGGGGSGGGGSEVQLVESGGGLVQAGGSLRLSCAASGITFSINTMGWYRQAPGKQRELVALISSIGDTYYADSVKGRFTISRDNAKNTVYLQMNSLKPEDTAVYYCKRFRTAAQGTDYWGQGTLVTVSSA

SEQ ID NO: 67 - protein, 431 aa, synthetic construct
  REGION 1..431: note = trivalent bispecific compound
DVQLVESGGGVVQPGGSLRLSCAASGSTISSYVMGWFRRAPGKEREFVAAISRSGGYTYYADSVKGRFTISRDNSKKTAYLQMNSLRPEDTALYYCAAGRYSAWYSQSYEYDYWGQGTLVTVSSGGGGSGGGGSGGGGSGGGGSGGGGSGGGGSGGGGSEVQLVESGGGLVQAGGSLRLSCAASGITFSINTMGWYRQAPGKQRELVALISSIGDTYYADSVKGRFTISRDNAKNTVYLQMNSLKPEDTAVYYCKRFRTAAQGTDYWGQGTLVTVSSGGGGSGGGGSGGGGSGGGGSGGGGSGGGGSGGGGSEVQLVESGGGLVQAGGSLRLSCAASGITFSINTMGWYRQAPGKQRELVALISSIGDTYYADSVKGRFTISRDNAKNTVYLQMNSLKPEDTAVYYCKRFRTAAQGTDYWGQGTLVTVSSA

SEQ ID NO: 68 - protein, 448 aa, synthetic construct
  REGION 1..448: note = trivalent compound
DVQLVESGGGVVQPGGSLRLSCAASGRTFSSYAMGWFRQAPGKEREFVAAISWSDGSTYYADSVKGRFTISRDNAKNTVYLQMNSLRPEDTALYYCAADLTSTNPGSYIYIWAYDYWGQGTLVTVSSGGGGSGGGGSGGGGSGGGGSGGGGSGGGGSGGGGSEVQLVESGGGVVQPGGSLRLSCAASGRTFSSYAMGWFRQAPGKEREFVAAISWSDGSTYYADSVKGRFTISRDNAKNTVYLQMNSLRPEDTALYYCAADLTSTNPGSYIYIWAYDYWGQGTLVTVSSGGGGSGGGGSGGGGSGGGGSGGGGSGGGGSGGGGSEVQLVESGGGVVQPGGSLRLSCAASGSTISSYVMGWFRRAPGKEREFVAAISRSGGYTYYADSVKGRFTISRDNSKKTAYLQMNSLRPEDTALYYCAAGRYSAWYSQSYEYDYWGQGTLVTVSS

SEQ ID NO: 69 - protein, 609 aa, Homo sapiens [sequence omitted]

SEQ ID NO: 70 - protein, 607 aa, Mus musculus
MKWVTFLLLLFVSGSAFSRGFRREAHKSEIAHRYNDLGEQHFKGLVLIAFSQYLQKCSYDEHAKLVQEVTDFAKTCVADESAANCDKSLHTLFGDKLCAIPNLRENYGELADCCTKQEPERNECFLQHKDDNPSLPPFERPEAEAMCTSFKENPTTFMGHYLHEVARRHPYFYAPELLYYAEQYNEILTQCCAEADKESCLTPKLDGVKEKALVSSVRQRMKCSSMQKFGERAFKAWAVARLSQTFPNADFAEITKLATDLTKVNKECCHGDLLECADDRAELAKYMCENQATISSKLQTCCDKPLLKKAHCLSEVEHDTMPADLPAIAADFVEDQEVCKNYAEAKDVFLGTFLYEYSRRHPDYSVSLLLRLAKKYEATLEKCCAEANPPACYGTVLAEFQPLVEEPKNLVKTNCDLYEKLGEYGFQNAILVRYTQKAPQVSTPTLVEAARNLGRVGTKCCTLPEDQRLPCVEDYLSAILNRVCLLHEKTPVSEHVTKCCSGSLVERRPCFSALTVDETYVPKEFKAETFTFHSDICTLPEKEKQIKKQTALAELVKHKPKATAEQLKTVMDDFAQFLDTCCKAADKDTCFSTEGPNLVTRCKDALA

SEQ ID NO: 71 - protein, 608 aa, Rattus norvegicus
MKWVTFLLLLFISGSAFSRGVFRREAHKSEIAHRFKDLGEQHFKGLVLIAFSQYLQKCPYEEHIKLVQEVTDFAKTCVADENAENCDKSIHTLFGDKLCAIPKLRDNYGELADCCAKQEPERNECFLQHKDDNPNLPPFQRPEAEAMCTSFQENPTSFLGHYLHEVARRHPYFYAPELLYYAEKYNEVLTQCCTESDKAACLTPKLDAVKEKALVAAVRQRMKCSSMQRFGERAFKAWAVARMSQRFPNAEFAEITKLATDLTKINKECCHGDLLECADDRAELAKYMCENQATISSKLQACCDKPVLQKSQCLAEIEHDNIPADLPSIAADFVEDKEVCKNYAEAKDVFLGTFLYEYSRRHPDYSVSLLLRLAKKYEATLEKCCAEGDPPACYGTVLAEFQPLVEEPKNLVKTNCELYEKLGEYGFQNAILVRYTQKAPQVSTPTLVEAARNLGRVGTKCCTLPEAQRLPCVEDYLSAILNRLCVLHEKTPVSEKVTKCCSGSLVERRPCFSALTVDETYVPKEFKAETFTFHSDICTLPDKEKQIKKQTALAELVKHKPKATEDQLKTVMGDFAQFVDKCCKAADKDNCFATEGPNLVARSKEALA

SEQ ID NO: 72 - protein, 607 aa, Bos taurus
MKWVTFISLLLLFSSAYSRGVFRRDTHKSEIAHRFKDLGEEHFKGLVLIAFSQYLQQCPFDEHVKLVNELTEFAKTCVADESHAGCEKSLHTLFGDELCKVASLRETYGDMADCCEKQEPERNECFLSHKDDSPDLPKLKPDPNTLCDEFKADEKKFWGKYLYEIARRHPYFYAPELLYYANKYNGVFQECCQAEDKGACLLPKIETMREKVLTSSARQRLRCASIQKFGERALKAWSVARLSQKFPKAEFVEVTKLVTDLTKVHKECCHGDLLECADDRADLAKYICDNQDTISSKLKECCDKPLLEKSHCIAEVEKDAIPENLPPLTADFAEDKDVCKNYQEAKDAFLGSFLYEYSRRHPEYAVSVLLRLAKEYEATLEECCAKDDPHACYSTVFDKLKHLVDEPQNLIKQNCDQFEKLGEYGFQNALIVRYTRKVPQVSTPTLVEVSRSLGKVGTRCCTKPESERMPCTEDYLSLILNRLCVLHEKTPVSEKVTKCCTESLVNRRPCFSALTPDETYVPKAFDEKLFTFHADICTLPDTEKQIKKQTALVELLKHKPKATEEQLKTVMENFVAFVDKCCAADDKEACFAVEGPKLVVSTQTALA

SEQ ID NO: 73 - protein, 607 aa, Sus scrofa
MKWVTFISLLFLFSSAYSRGVFRRDTYKSEIAHRFKDLGEQYFKGLVLIAFSQHLQQCPYEEHVKLVREVTEFAKTCVADESAENCDKSIHTLFGDKLCAIPSLREHYGDLADCCEKEEPERNECFLQHKNDNPDIPKLKPDPVALCADFQEDEQKFWGKYLYEIARRHPYFYAPELLYYAIIYKDVFSECCQAADKAACLLPKIEHLREKVLTSAAKQRLKCASIQKFGERAFKAWSLARLSQRFPKADFTEISKIVTDLAKVHKECCHGDLLECADDRADLAKYICENQDTISTKLKECCDKPLLEKSHCIAEAKRDELPADLNPLEHDFVEDKEVCKNYKEAKHVFLGTFLYEYSRRHPDYSVSLLLRIAKIYEATLEDCCAKEDPPACYATVFDKFQPLVDEPKNLIKQNCELFEKLGEYGFQNALIVRYTKKVPQVSTPTLVEVARKLGLVGSRCCKRPEEERLSCAEDYLSLVLNRLCVLHEKTPVSEKVTKCCTESLVNRRPCFSALTPDETYKPKEFVEGTFTFHADLCTLPEDEKQIKKQTALVELLKHKPHATEEQLRTVLGNFAAFVQKCCAAPDHEACFAVEGPKFVIEIRGILA

SEQ ID NO: 74 - protein, 608 aa, Canis lupus
MKWVTFISLFFLFSSAYSRGLVRREAYKSEIAHRYNDLGEEHFRGLVLVAFSQYLQQCPFEDHVKLAKEVTEFAKACAAEESGANCDKSLHTLFGDKLCTVASLRDKYGDMADCCEKQEPDRNECFLAHKDDNPGFPPLVAPEPDALCAAFQDNEQLFLGKYLYEIARRHPYFYAPELLYYAQQYKGVFAECCQAADKAACLGPKIEALREKVLLSSAKERFKCASLQKFGDRAFKAWSVARLSQRFPKADFAEISKVVTDLTKVHKECCHGDLLECADDRADLAKYMCENQDSISTKLKECCDKPVLEKSQCLAEVERDELPGDLPSLAADFVEDKEVCKNYQEAKDVFLGTFLYEYARRHPEYSVSLLLRLAKEYEATLEKCCATDDPPTCYAKVLDEFKPLVDEPQNLVKTNCELFEKLGEYGFQNALLVRYTKKAPQVSTPTLVEVSRKLGKVGTKCCKKPESERMSCAEDFLSVVLNRLCVLHEKTPVSERVTKCCSESLVNRRPCFSGLEVDETYVPKEFNAETFTFHADLCTLPEAEKQVKKQTALVELLKHKPKATDEQLKTVMGDFGAFVEKCCAAENKEGCFSEEGPKLVAAAQAALV

SEQ ID NO: 75 - protein, 608 aa, Felis catus
MKWVTFISLLLLFSSAYSRGVTRREAHQSEIAHRFNDLGEEHFRGLVLVAFSQYLQQCPFEDHVKLVNEVTEFAKGCVADQSAANCEKSLHELLGDKLCTVASLRDKYGEMADCCEKKEPERNECFLQHKDDNPGFGQLVTPEADAMCTAFHENEQRFLGKYLYEIARRHPYFYAPELLYYAEEYKGVFTECCEAADKAACLTPKVDALREKVLASSAKERLKCASLQKFGERAFKAWSVARLSQKFPKAEFAEISKLVTDLAKIHKECCHGDLLECADDRADLAKYICENQDSISTKLKECCGKPVLEKSHCISEVERDELPADLPPLAVDFVEDKEVCKNYQEAKDVFLGTFLYEYSRRHPEYSVSLLLRLAKEYEATLEKCCATDDPPACYAHVFDEFKPLVEEPHNLVKTNCELFEKLGEYGFQNALLVRYTKKVPQVSTPTLVEVSRSLGKVGSKCCTHPEAERLSCAEDYLSVVLNRLCVLHEKTPVSERVTKCCTESLVNRRPCFSALQVDETYVPKEFSAETFTFHADLCTLPEAEKQIKKQSALVELLKHKPKATEEQLKTVMGDFGSFVDKCCAAEDKEACFAEEGPKLVAAAQAALA

SEQ ID NO: 76 - protein, 607 aa, Ovis aries
MKWVTFISLLLLFSSAYSRGVFRRDTHKSEIAHRFNDLGEENFQGLVLIAFSQYLQQCPFDEHVKLVKELTEFAKTCVADESHAGCDKSLHTLFGDELCKVATLRETYGDMADCCEKQEPERNECFLNHKDDSPDLPKLKPEPDTLCAEFKADEKKFWGKYLYEVARRHPYFYAPELLYYANKYNGVFQECCQAEDKGACLLPKIDAMREKVLASSARQRLRCASIQKFGERALKAWSVARLSQKFPKADFTDVTKIVTDLTKVHKECCHGDLLECADDRADLAKYICDHQDALSSKLKECCDKPVLEKSHCIAEVDKDAVPENLPPLTADFAEDKEVCKNYQEAKDVFLGSFLYEYSRRHPEYAVSVLLRLAKEYEATLEDCCAKEDPHACYATVFDKLKHLVDEPQNLIKKNCELFEKHGEYGFQNALIVRYTRKAPQVSTPTLVEISRSLGKVGTKCCAKPESERMPCTEDYLSLILNRLCVLHEKTPVSEKVTKCCTESLVNRRPCFSDLTLDETYVPKPFDEKFFTFHADICTLPDTEKQIKKQTALVELLKHKPKATDEQLKTVMENFVAFVDKCCAADDKEGCFVLEGPKLVASTQAALA

SEQ ID NO: 77 - protein, 607 aa, Equus caballus
MKWVTFVSLLFLFSSAYSRGVLRRDTHKSEIAHRFNDLGEKHFKGLVLVAFSQYLQQCPFEDHVKLVNEVTEFAKKCAADESAENCDKSLHTLFGDKLCTVATLRATYGELADCCEKQEPERNECFLTHKDDHPNLPKLKPEPDAQCAAFQEDPDKFLGKYLYEVARRHPYFYGPELLFHAEEYKADFTECCPADDKLACLIPKLDALKERILLSSAKERLKCSSFQNFGERAVKAWSVARLSQKFPKADFAEVSKIVTDLTKVHKECCHGDLLECADDRADLAKYICEHQDSISGKLKACCDKPLLQKSHCIAEVKEDDLPSDLPALAADFAEDKEICKHYKDAKDVFLGTFLYEYSRRHPDYSVSLLLRIAKTYEATLEKCCAEADPPACYRTVFDQFTPLVEEPKSLVKKNCDLFEEVGEYDFQNALIVRYTKKAPQVSTPTLVEIGRTLGKVGSRCCKLPESERLPCSENHLALALNRLCVLHEKTPVSEKITKCCTDSLAERRPCFSALELDEGYVPKEFKAETFTFHADICTLPEDEKQIKKQSALAELVKHKPKATKEQLKTVLGNFSAFVAKCCGREDKEACFAEEGPKLVASSQLALA

SEQ ID NO: 78 - protein, 608 aa, Oryctolagus cuniculus
MKWVTFISLLFLFSSAYSRGVFRREAHKSEIAHRFNDVGEEHFIGLVLITFSQYLQKCPYEEHAKLVKEVTDLAKACVADESAANCDKSLHDIFGDKICALPSLRDTYGDVADCCEKKEPERNECFLHHKDDKPDLPPFARPEADVLCKAFHDDEKAFFGHYLYEVARRHPYFYAPELLYYAQKYKAILTECCEAADKGACLTPKLDALEGKSLISAAQERLRCASIQKFGDRAYKAWALVRLSQRFPKADFTDISKIVTDLTKVHKECCHGDLLECADDRADLAKYMCEHQETISSHLKECCDKPILEKAHCIYGLHNDETPAGLPAVAEEFVEDKDVCKNYEEAKDLFLGKFLYEYSRRHPDYSVVLLLRLGKAYEATLKKCCATDDPHACYAKVLDEFQPLVDEPKNLVKQNCELYEQLGDYNFQNALLVRYTKKVPQVSTPTLVEISRSLGKVGSKCCKHPEAERLPCVEDYLSVVLNRLCVLHEKTPVSEKVTKCCSESLVDRRPCFSALGPDETYVPKEFNAETFTFHADICTLPETERKIKKQTALVELVKHKPHATNDQLKTVVGEFTALLDKCCSAEDKEACFAVEGPKLVESSKATLG